(19) United States
(12) Patent Application Publication
(10) Pub. No.: US 2025/0237616 A1
(43) Pub. Date: Jul. 24, 2025

(54) Programmable Multi-Wavelength RF Spectrometry Networks

(71) Applicant: The Regents of the University of California, Oakland, CA (US)

(72) Inventors: Peter Tseng, Irvine, CA (US); Manik Dautta, Irvine, CA (US); Amirhossein Hajiaghajani, Irvine, CA (US)

(21) Appl. No.: 18/848,426

(22) PCT Filed: Mar. 19, 2023
(86) PCT No.: PCT/US2023/015590

§ 371 Date: Sep. 18, 2024

Related U.S. Application Data
(60) Provisional application No. 63/321,750, filed on Mar. 20, 2022.

(51) Int. Cl. G01N 22/00; H04L 67/12; H04W 4/38; H04W 88/04

(52) U.S. Cl. CPC G01N 22/00; H04L 67/12; H04W 4/38; H04W 88/04

(57) ABSTRACT

Programmable multi-wavelength RF spectrometry networks in accordance with embodiments of the invention are disclosed. In one embodiment, a sensor network for spectrometric monitoring of environmental signals is provided, the network comprising: an RF reader wireless connected to an intermediate relay; the intermediate relay coupled to a plurality of passive radio frequency (RF) sensors; wherein the plurality of RF sensors are individually programmable for measuring at least one environmental signal.

### CROSS-REFERENCE TO RELATED APPLICATION

The current application claims priority to U.S. Provisional Patent Application No. 63/321,750 filed on Mar. 20, 2022, the disclosure of which is incorporated herein by reference.

### FEDERAL FUNDING SUPPORT

This invention was made with Government support under Grant No. ECCS-1942364, awarded by the National Science Foundation. The Government has certain rights in the invention.

### FIELD OF THE INVENTION

The present disclosure generally relates to sensors and more specifically to programmable multi-wavelength RF spectrometry of various environments using adaptable networks of flexible and environmentally-responsive, passive wireless elements.

### BACKGROUND

Spectrometric readout has wide uses in modern science, where electromagnetic (EM) radiation of varying frequencies can be used to probe matter in its varying forms (whether simple or complex). For example, spectral signatures that manifest from such EM interactions may be used as an important identification tool in chemistry, physics, and biomedicine. A major characteristic of this readout is its data-rich nature, which enables a significant amount of information to be extracted in a single measurement. This manifests from the differential response of matter to varying EM wavelengths that may span x-rays, UV-Vis, terahertz, and radio-frequency. Some of the most widely-utilized methods include a material-under-test that is excited directly by a broad spectrum of radiation—examples include FTIR/UV-vis and mass spectrometry, wherein characteristic peaks over a broad spectrum yield rich, multiparametric data on the material-under-test.

### SUMMARY OF THE INVENTION

The various embodiments of the present programmable multi-wavelength RF spectrometry networks have several features, no single one of which is solely responsible for their desirable attributes. Without limiting the scope of the present embodiments as expressed by the claims that follow, their more prominent features now will be discussed briefly. After considering this discussion, and particularly after reading the section entitled “Detailed Description,” one will understand how the features of the present embodiments provide the advantages described herein.

One aspect of the present embodiments includes the realization that modern processing techniques enable a more focused approach, wherein matter is organized into structures that interact with radiation in a directed way, such as to exhibit resonance phenomena. This approach may be used to create more selective/sensitive sensors that are typically intended to measure a single value. A common example of this are RF sensors, wherein conductive traces are patterned so as to resonate when excited by RF waves. Such an approach may be adapted to build sensors and biosensors sensitive to a variety of chemophysical signals, such as pressure, temperature, glucose, salinity, nutrients, and more. Despite the emerging versatility of this approach, RF sensor readout is still highly limited, as typically only a single sensor is assessed at a time, and the technique is not stable to mechanical noise because readout coil and sensor alignment are typically not fixed.

As further described below, the present embodiments include a form of programmable RF spectrometry, wherein a single readout of RF spectra may be used to assess a wide-variety of desired chemophysical signals from the environment. This is in contrast to standard readout of multiparametric signals where individual sensing formats typically require unique signal conditioning circuitry and/or processing. In the present embodiments, RF waves may interact with multilayers of electronics-free patterned, wirelessly-coupled elements that may be engineered to various length-scales, to deform or attach around surfaces, and tuned to controlled reactivity to chemical and/or physical signals. As further dissuced below, the present embodiments are a broad expansion of more basic iterations of techniques, that monitors arrays of pressure sensors via planar readout coils, or the radiation of an array of temperature sensors. In the present embodiments, RF signal may first be mediated by passive intermediate relay coils that may be wireless and electrically-disconnected from other elements. This can transfer signal over intermediate distances and can be fused onto textiles or conform over surfaces. These relays may then be wirelessly-coupled to RF sensors with tunable environmental reactivity—demonstrated herein include pressure, temperature, salinity, and nutrients (sugars/salts/fats). This may then form a multiparametric network composed exclusively of passive material architectures. Beyond the fully passive/wireless co-readout of multiparametric signals, the present embodiments are significantly more robust in comparison to traditional RF readout—this is because intermediate coil to RF sensor alignment may be readily remain fixed through design. In general, any capacitive or resistive sensor type may be integrated with the present embodiments, as these readily build into RF sensors such as those disscussed herein. As further described below, the present embodiments demonstrate multiparametric, chemophysical readouts from wireless wristbands and cups (e.g., so called “SmartCups”) that are infused with multi-layers of interacting, flexible/reactive wireless elements.

The present embodiments demonstrate an adaptable, passive wireless sensor networks composed exclusively of material architectures without any electronic components. In many embodiments, intermediate relays allow signals to transmit across longer distances and over curved surfaces, while individually-placed passive wireless sensors along the network enable the co-monitoring of chemical and/or physical signals. Such strategies resolve many traditional issues hampering both electronically-mediated and passive wireless sensor readout. A single readout enables complex multiparametric signal extraction without any unique circuitry. Additionally, this network readout is robust to mechanical perturbation (a major issue with standard readout), and the IR allows the network to span across unique environments such as the body or utensils. In addition, fabrication techniques utilized allow the integration of network components into a multitude of environments, such as textiles, curved surfaces, and more. Such strategies may become the cornerstone of next-generation sensor networks that require no microelectronic components.

### BRIEF DESCRIPTION OF THE DRAWINGS

The various embodiments of the present programmable multi-wavelength RF spectrometry networks now will be discussed in detail with an emphasis on highlighting the advantageous features. These embodiments depict the novel and non-obvious programmable multi-wavelength RF spectrometry networks shown in the accompanying drawings, which are for illustrative purposes only. These drawings include the following figures:

FIG. 1A illustrates a network comprising multi-layers of passive (zero-electronic) elements enabling a single readout co-monitoring of complex signals in accordance with an embodiment of the invention.

FIG. 1B is a perspective view of flexible IR integrated on an outer surface of cups in accordance with an embodiment of the invention.

FIG. 1C illustrates an IR-integrated smart textile to facilitate multiparametric wristband readouts in accordance with an embodiment of the invention.

FIG. 1D illustrates readout antenna structures in accordance with an embodiment of the invention.

FIG. 1E are graphs illustrating spectral readout from various network configurations in accordance with an embodiment of the invention.

FIG. 2A illustrates effects of alignment between a readout antenna and IR when IR and sensor orientation is fixed in accordance with an embodiment of the invention.

FIG. 2B illustrates effects of alignment between IR and sensor while antenna and IR are fixed in accordance with an embodiment of the invention.

FIG. 2C illustrates readout of multiple sensors through multiple IR in accordance with an embodiment of the invention.

FIG. 2D illustrates readout of multiple sensors by IR in accordance with an embodiment of the invention.

FIG. 3A illustrates effect of positional coupling between sensors in accordance with an embodiment of the invention.

FIG. 3B illustrates effect of sensimetric coupling between sensors (applied pressure) in accordance with an embodiment of the invention.

FIG. 4A illustrates multiparametric readout from a wearable wristband in accordance with an embodiment of the invention.

FIG. 4B illustrates spectrometric co-readout of sensor state in accordance with an embodiment of the invention.

FIG. 4C illustrates a Smartcup for co-monitoring nutrients in a drink in accordance with an embodiment of the invention.

FIG. 4D illustrates spectrometric co-readout of sensors state in accordance with an embodiment of the invention.

FIG. 5A-C illustrate readout coil geometry of (a) CWOG, (b) CWG, and (c) PWG readout antennas in accordance with an embodiment of the invention.

FIGS. 6A-B illustrate IR integrated smart textile including (a) flexible IR on the textile, (b) placement of the Wristband and portable NanoVNA to read out multiparametric sensor state in accordance with an embodiment of the invention.

FIG. 7 illustrates a 3.25 turns spiral square trilayer sensor structure where two spiral resonators were interceded by Ecoflex 10 in accordance with an embodiment of the invention.

FIG. 8 illustrates effect of vertical distance between the sensor and the readout coil in accordance with an embodiment of the invention.

FIG. 9A illustrates geometry of a sensor and readout coil used in the simulation in accordance with an embodiment of the invention.

FIG. 9B illustrates spectral response of a sensor by simulation (dash line), and by experiment (solid line) in accordance with an embodiment of the invention.

FIG. 9C illustrates E-field distribution of a sensor due to CWOG and CWG readout coils in accordance with an embodiment of the invention.

FIG. 9D illustrates H-field distribution of a sensor due to CWOG and CWG readout coils illustrates in accordance with an embodiment of the invention.

FIG. 10 illustrates effect of bends in the IR on the measured spectral response of the sensor in accordance with an embodiment of the invention.

FIG. 11 illustrates alignment between IR and antenna while sensor and IR placement is fixed in accordance with an embodiment of the invention.

FIG. 12 illustrates effects of alignment between readout coil and the sensor without IR in accordance with an embodiment of the invention.

FIGS. 13A-B illustrate length study: a) effect of small changes in the length, b) large changes in the length in accordance with an embodiment of the invention.

FIG. 14 illustrates positional coupling effect among sensors measured by one-by-one removal above a CWOG antenna in accordance with an embodiment of the invention.

FIG. 15 illustrates positional coupling effect among sensors measured by one-by-one removal above a CWG antenna in accordance with an embodiment of the invention.

FIGS. 16A-B illustrate position coupling effect among sensors with an IR30/50: (a) Schematic of the readout coil, IR, and sensors, alongside network spectral response with (b) CWOG or (c) CWG in accordance with an embodiment of the invention.

FIG. 17 illustrates coupling among sensors due to individual sensor perturbation (pressure) in CWOG in accordance with an embodiment of the invention.

FIG. 18 illustrates coupling among sensors due to individual sensor perturbation (pressure) in CWOG in accordance with an embodiment of the invention.

FIG. 19 illustrates positional coupling effect among sensors measured by one-by-one removal above a PWG antenna in accordance with an embodiment of the invention.

FIG. 20 illustrates coupling among sensors due to individual sensor perturbation (pressure) in PWG in accordance with an embodiment of the invention.

FIGS. 21A-C illustrate coupling among sensors due to individual sensor perturbation (pressure) with an interceding IR: (a) schematic of the placement of the readout coil, IR, and sensors, and network spectral response for (b) CWOG, or (c) CWG in accordance with an embodiment of the invention.

FIGS. 22A-D illustrate sensors used in wristband: (a) PEG-1500 interlayer temperature sensor, (b) Ecoflex-10 interlayer pressure sensor, (c) PEGDA700 hydrogel interlayer salt sensor, and (d) p (NIPAM-AA) hydrogel interlayer pH sensor in accordance with an embodiment of the invention.

FIGS. 23A-D illustrate sensors in SmartCup: (a) PEG-1500 interlayer temperature sensor, (b) nonporous silk fibroin interlayer salt sensor, (c) nonporous silk fibroin interlayer sugar sensor, and (d) porous silk fibroin interlayer fat sensor in accordance with an embodiment of the invention.

### DETAILED DESCRIPTION OF THE DRAWINGS

The various embodiments of the present programmable multi-wavelength RF spectrometry networks contain several features, no single one of which is solely responsible for their desirable attributes. Without limiting the scope of the present embodiments, their more prominent features will now be discussed below. In particular, the present programmable multi-wavelength RF spectrometry networks will be discussed in the context of specific sensors and chemophysical environments. However, the use of specific sensors and chemophysical environments are merely exemplary as various sensors may be utilized for various environments as appropriate to the requirements of a specific application in accordance with embodiments of the invention. Further, the particular use of any number of sensors and network parameters are exemplary, and various other suitable type and number of sensors and network parameters may be appropriate to the requirements of a specific application in accordance with various embodiments of the invention. After considering this discussion, and particularly after reading the section entitled “Detailed Description,” one will understand how the features of the present embodiments provide the advantages described here.

The following detailed description describes the present embodiments with reference to the drawings. In the drawings, reference numbers label elements of the present embodiments. These reference numbers are reproduced below in connection with the discussion of the corresponding drawing features. These figures, and their written descriptions, indicate that certain components of the apparatus are formed integrally, and certain other components are formed as separate pieces. Those of ordinary skill in the art will appreciate that components shown and described herein as being formed integrally may in alternative embodiments be formed as separate pieces. Those of ordinary skill in the art will further appreciate that components shown and described herein as being formed as separate pieces may in alternative embodiments be formed integrally. Further, as used herein the term integral describes a single unitary piece.

Turning now to the drawings, readout of multiparametric environmental signals typically uses discrete sensing formats that individually require unique signal conditioning circuitry and/or processing pathways. The present embodiments utilize adaptable sensor networks composed exclusively of passive material architectures that enable spectrometric co-monitoring of chemical and/or physical environmental signals. As further described below, a single RF reader may wirelessly interact first with an intermediate wireless relay coil (may also be referred to as “relay” or “IR”) which may be tunable in length and may be configured to conform around surfaces. In many embodiments, the relay (which may be fused on textiles and/or surfaces) may then wirelessly be coupled to a plurality of passive RF sensors (e.g., arrays of passive RF sensors) with individually programmable flexibility/reactivity to environmental signals. In various embodiments, multiple chemical and/or physical signals may then be monitored within the single spectral readout of a wearable reader. This technique may probe over tunable length scales, and may be robust to mechanical disturbances that limit conventional techniques. As further described below, the present embodiments may be used to co-monitor chemophysical metrics such as, but not limited to, nutrients, temperature, pressure, pH, and more on the skin or in utensils with a single readout. The present embodiments may form a cornerstone of zero-microelectronic sensor networks. Programmable multi-wavelength RF spectrometry networks (may also be referred to as “networks” or “passive wireless networks”) accordance with embodiments of the invention are further discussed below.

### Programmable Multi-Wavelength RF Spectrometry Networks

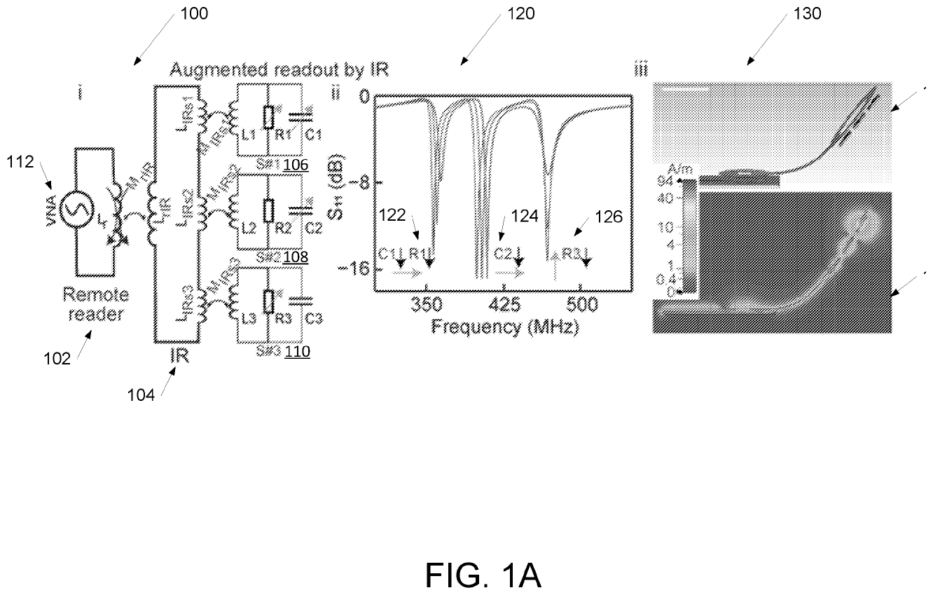

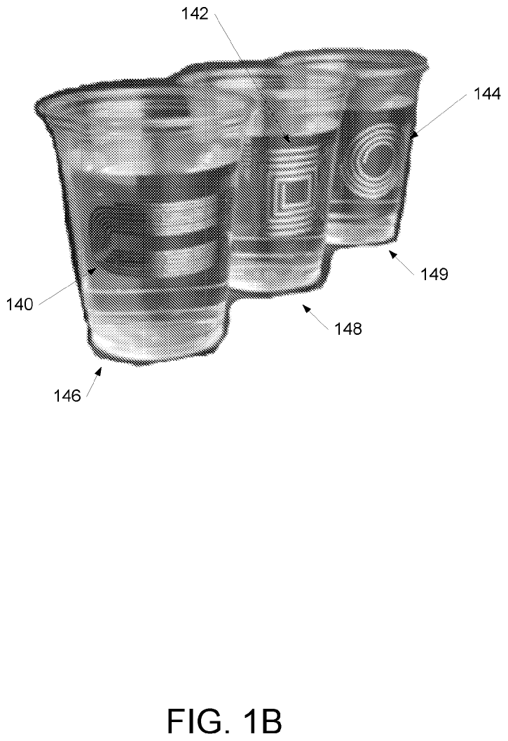

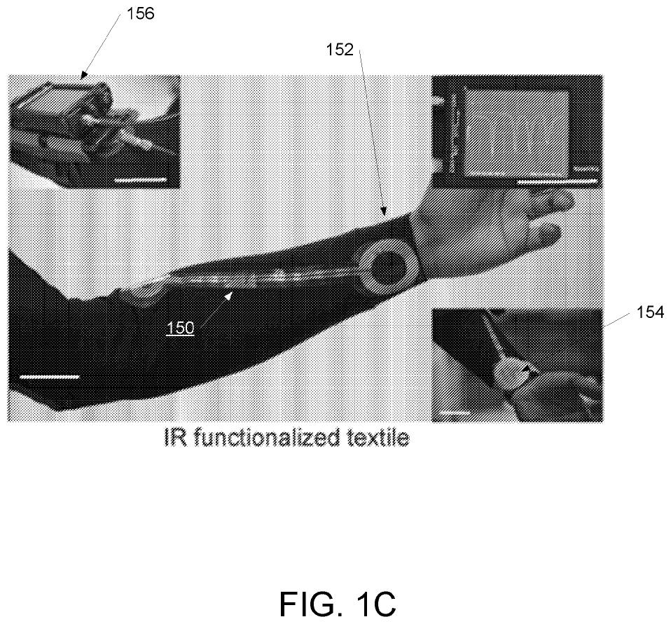

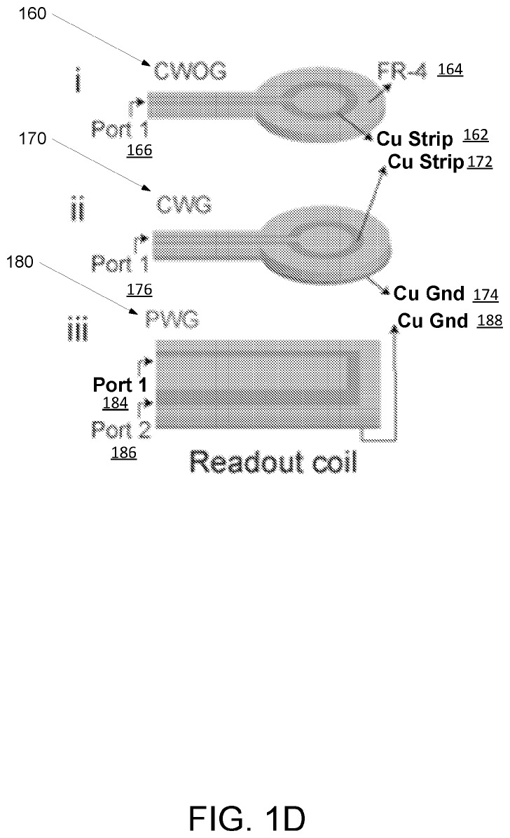

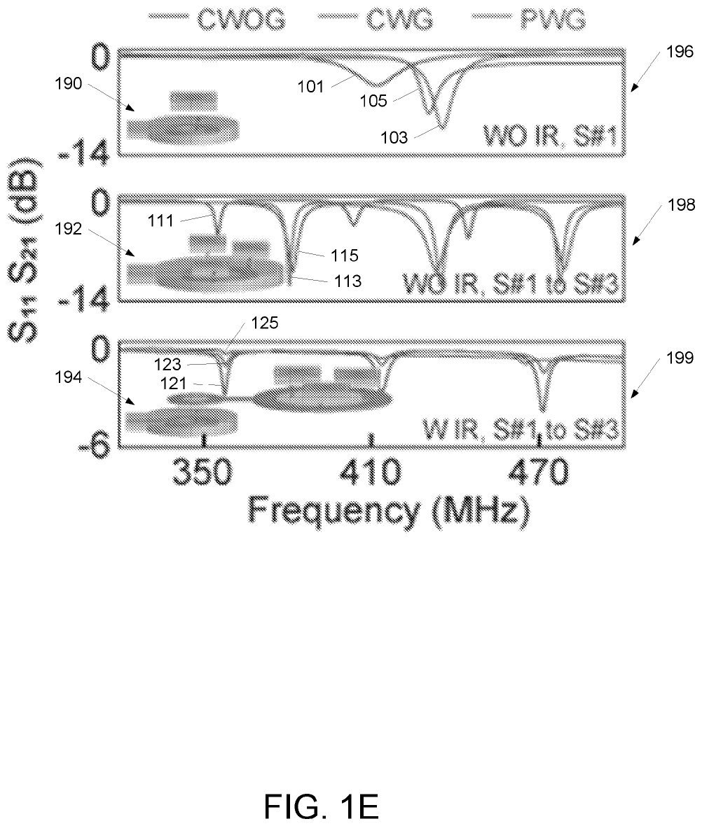

A programmable multi-wavelength RF spectrometry may be utilized in various environments including, but not limited to, a chemophysical environment. A programmable multi-wavelength RF spectrometry of the chemophysical environment is illustrated. A network comprising multi-layers of passive (zero-electronic) elements enabling a single readout co-monitoring of complex signals in accordance with an embodiment of the invention is shown in FIG. 1A. FIG. 1A (i) 100 includes a circuit diagram of reader 102 wirelessly-coupled to an intermediate relay (IR) 104, in turn wirelessly-coupled to tunable RF sensors 106, 108, 110.

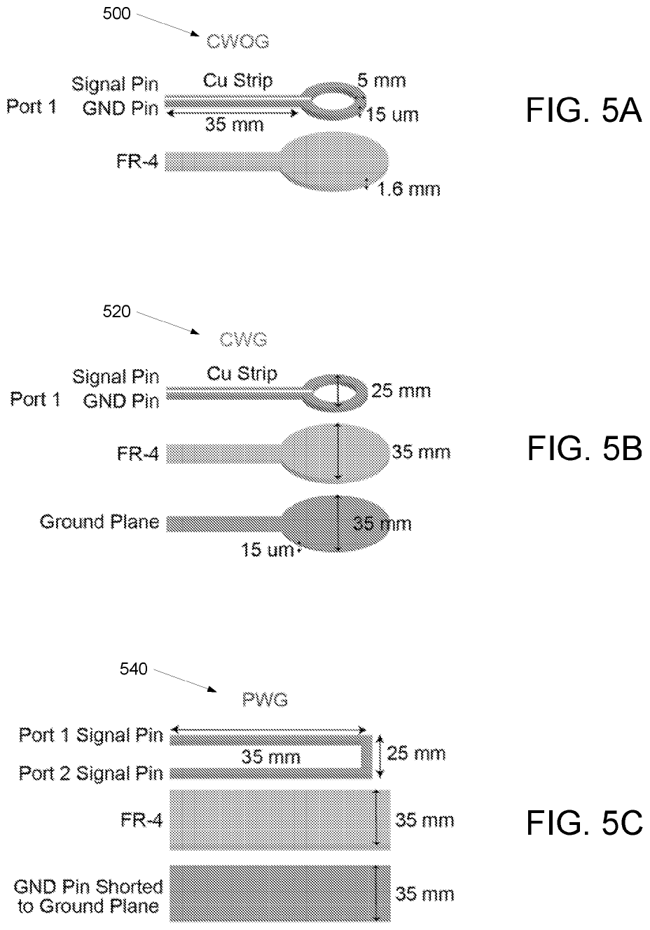

In reference to FIG. 1A, an approach may include 3 types of RF elements 106, 108, 110, that are wirelessly coupled to form the complete circuit as shown in FIG. 1A (i). In many embodiments, the networks may include readout coils that may form the initial inductive link into a passive sensor network, and that is probed via direct wired connection to a reader 102 (such as, but not limited to, a tabletop or wearable VNA 112). The inductive readout coil may be configured as a one port circular coil for S₁₁ or a two port microstrip patch line for 6₁ spectral response readout (see FIG. 5A-C which illustrate readout coil geometry of (a) CWOG 500, (b) CWG 520, and (c) PWG 540 readout antennas). Herein, the network may utilize either a 25 mm diameter circular readout coil (feed line length 35 mm) or U-shaped microstrip patch line (25 and 35 mm traces) that may be selectively integrated with a FR-4 substrate (fabrication of which is discussed below). In various embodiments, the networks may also include an intermediate relay (IR) coil that may be untethered from all other elements. This is wirelessly coupled with the readout coil, transferring the EM fields to subsequent sensors along its pathlength or through designed inductive terminals. This IR may play an important role in the structure—in varous embodiments, it is synthesized on flexible substrate, and subsequently fused onto curved surfaces or textiles. This allows RF signal to transmit over materials/substrates relevant to daily life, and may be tuned to transfer signal over arbitrary distances. Beyond facilitating information from localized sensing nodes, these enhance the mechanical robustness of the sensing network. Sensor alignment to intermediate coil is relatively simple to maintain due to the flexible/routing nature of the IR—as further described below, this helps stabilize the spectral readout to misalignment between the readout coil and network. This adds significant flexibility to the final passive sensor network.

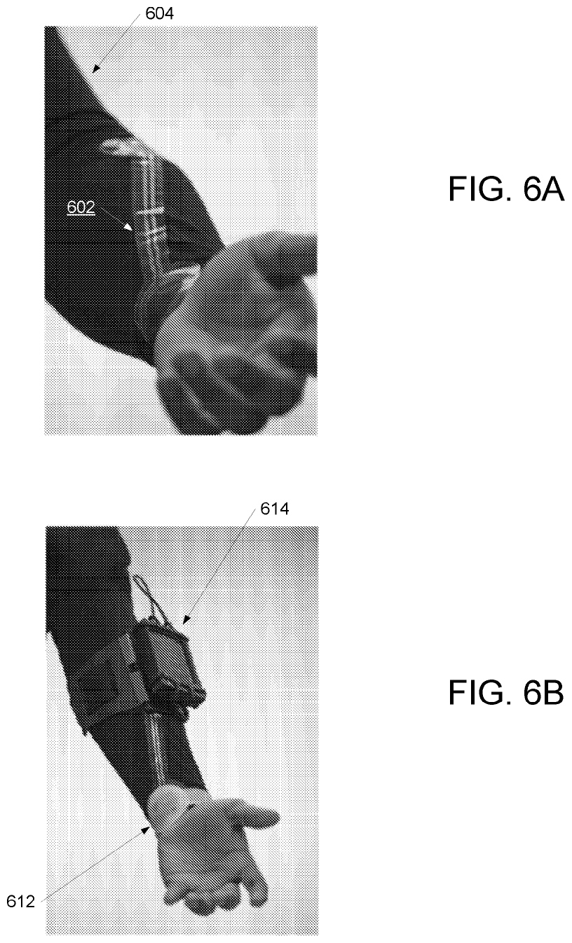

A perspective view of flexible IRs 140, 142, 144 integrated on an outer surface of cups 146, 148, 149, respectively in accordance with an embodiment of the invention is shown in FIG. 1B. An IR-integrated smart textile 150 to facilitate multiparametric wristband readouts in accordance with an embodiment of the invention is shown in FIG. 1C. The present embodiments demonstrate various practical manifestations wherein the IR coil (e.g., IR coils 140, 142, 144) is embedded alongside a cup (e.g., 146, 148, 149) to enable a SmartCup for co-monitoring nutrients in food (as shown in FIG. 1B), or fused on a textile (e.g., arm sleeve 152) to facilitate readout of a wristband from across the arm (as shown in FIG. 1C, and FIG. 6, which illustrate IR integrated smart textile including (a) flexible IR 602 on the textile 604, (b) placement of the Wristband 612 and portable NanoVNA 614 to read out multiparametric sensor state). In reference to FIG. 1C, insets may be the placement of the wristband 154 and direct readout from wearable NanoVNA 156.

In many embodiments, the networks may include passive and wireless RF sensors with individually-tunable mechanical or chemical reactivity. These sensors may be passive RLC structures that are built to modulate with environmental signals. As described herein, the present embodiments utilize broadside coupled, split ring resonating architectures that have been previously characterized. One key aspect of the strategy of the present embodiments is the utilization of interlayer-RF sensor design schemes. Modulation of the lumped resistance of a sensor changes the magnitude, while modulation of the lumped capacitance shifts the resonant frequency of its spectral response. Individual sensors may be built with specialized materials (both within and around the sensing architecture) and thus rendered selectively-sensitive to metrics such as, but not limited to, glucose, sugars, salts, fats, pressure, temperature, and more. Importantly, these structures may be readily tuned to respond/resonate at different wavelengths, and thus occupy individual frequency bands during spectral readout. This occurs by simply varying the thickness of the interlayer. This allows the networks to readily tune any sensor of a set square area (size footprint) to hit variable operating frequencies. Thus for various sensors (e.g., sensors that are 0.5 to 1 cm wide), the present embodiments could readily tune response to occupy various desired bands for different environmental responses. In some embodiments, these sensors may be oriented along the IR coil, and whose resonance can be probed through the intermediate relay signal.

FIG. 1A (ii) 120 includes an RF simulation of the spectral readout where sensor 1 is perturbed 122 by both R1 and C1, sensor 2 is perturbed 124 only in C2, and sensor 3 is perturbed 126 only in R3. FIG. 1A (iii) 130 includes geometry used in FEM (top) 132 and corresponding magnetic field distribution 134 showing magnetic coupling between elements. The final, versatile structure is a fully passive sensor network (requiring zero electronics) that may monitor complex chemophysical signals in a single readout. FIG. 1A (ii) 120 shows the RF simulation of co-readout of three sensors (numbered S#1 106, S#2 108, and S#3 110, respectively). Here, both C1 and R1 of S#1, only C2 of S#2, and only R3 of S#3 are perturbed. Enlargement of R3 decreases the signal magnitude, reduction in C2 increases the resonant frequency, while reductions in both R1 and C1 decreases the signal magnitude and increases the resonant frequency respectively. These modulations map exclusively to the spectral band occupied by individual sensors. FIG. 1A (iii) 130 shows a finite element simulation of the magnetic fields 134 within a sample network. This field distribution displays the multiple layers of wireless magnetic coupling between readout coil and sensors via the IR. This system exhibits additional power loss in comparison to traditional RF sensor readout due to the additional wireless couplings—specifically the coupling between readout coil and IR, and coupling between IR and sensors. The effect of this interceding coil can be seen in the reduced magnitude S₁₁ response of the multi-coil network as opposed to the direct readout of sensors (this is for the same input dbm to both configurations). The impact of the lower S₁₁ is that shifts in the magnitude and frequency of resonant sensors may become more difficult to resolve. A higher power may be used to increase the total S₁₁ response, and thus improve the readout of very low-sensitivity sensors, but, there may be an upper limit to the total power that may be input in wearable, or close-to-body applications. Thus, in networks using an IR typically require moderate to high-sensitivity sensors in near-body environments. However, it is noted that this type of moderate to high-sensitivity is not difficult, as all of the demonstrated sensors herein are easily probed/measured with −5 dbm (˜300 mW), which is standard for many wearable applications in nearfield.

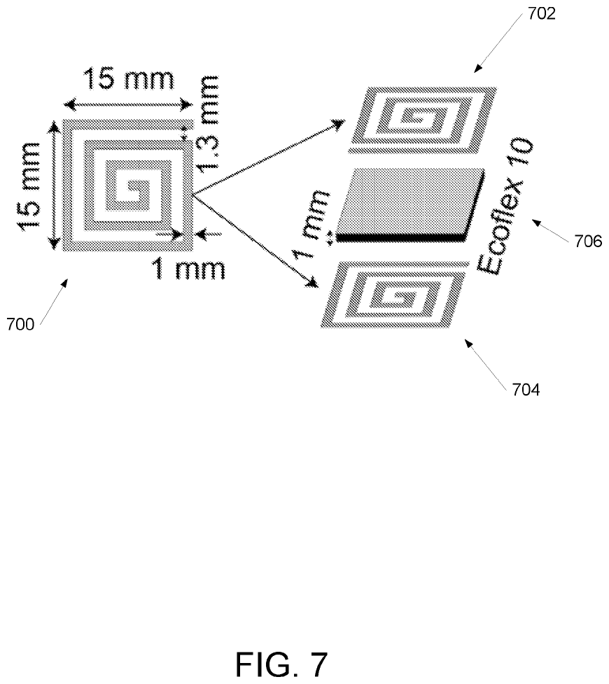

Readout antenna structures in accordance with an embodiment of the invention is shown in FIG. 1D. By way of example, the present embodiments provide three readout antennas for targeting different applications: Circular Without Ground plane (CWOG) 160, Circular With Ground plane (CWG) 170, and Patch With Ground plane (PWG) 180 as shown in FIG. 1D. The CWOG 160 may be a circular loop readout coil 162 pasted on FR-4 substrate 164 which has one port 166 connected to the VNA, whereas the CWG 170 may be the same readout coil 172 but the other side of FR-4 substrate has a conductive ground plane 174, which has one port 176 connected to a VNA. PWG 180 may be a microstrip patch line 182 which has two ports 184, 186 connected to the VNA, and the common ground pin is shorted via the connection with the ground plane 188 on the other side of the FR-4 substrate. A detailed layout is presented in FIGS. 5A-C. Sensors may be variations of interlayer-RF structures, of which the fundamental structure was a 15-mm-wide, 3.25 turn spiral square trilayer structure (see FIG. 7, which illustrates a 3.25 turns spiral square trilayer sensor structure 700 where two spiral resonators 702, 704 were interceded by Ecoflex 10 706). This structure may be modulated in several ways to broadly tune the sensor to different resonant frequencies while retaining the same footprint: via modification of the coil turn number or interlayer thickness.

Graphs illustrating spectral readout from various network configurations in accordance with an embodiment of the invention is shown in FIG. 1E. FIG. 1E illustrates spectral readouts 196, 198, 199 from various network configurations: single sensor 190 without IR (diameter of the readout coil loop is 25 mm), multiple sensors 192 without IR (diameter of the readout coil loop is 50 mm), and multiple sensors 194 with IR (diameter of the IR loop is 50 mm). In many embodiments, the standard vertical distance between readout coil/IR loop and sensors are 0.5 mm. For multisensor readout, the orientation of the sensors may remain constant (scale bars are 5 cm).

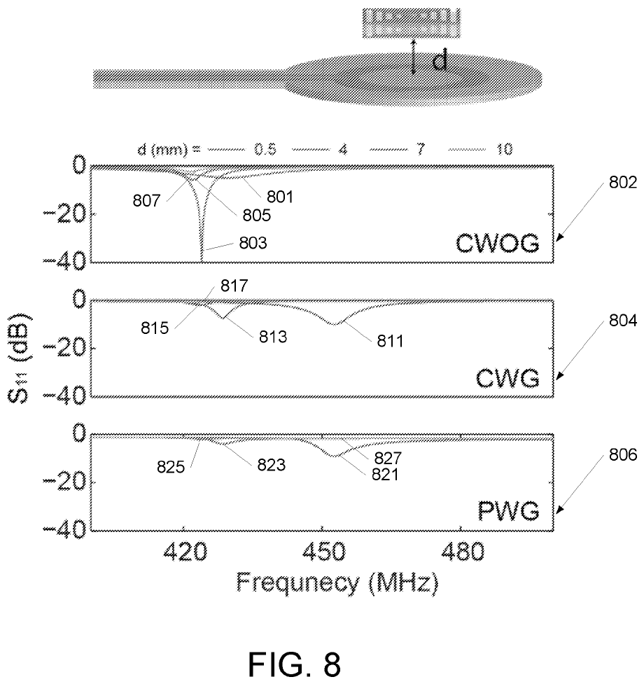

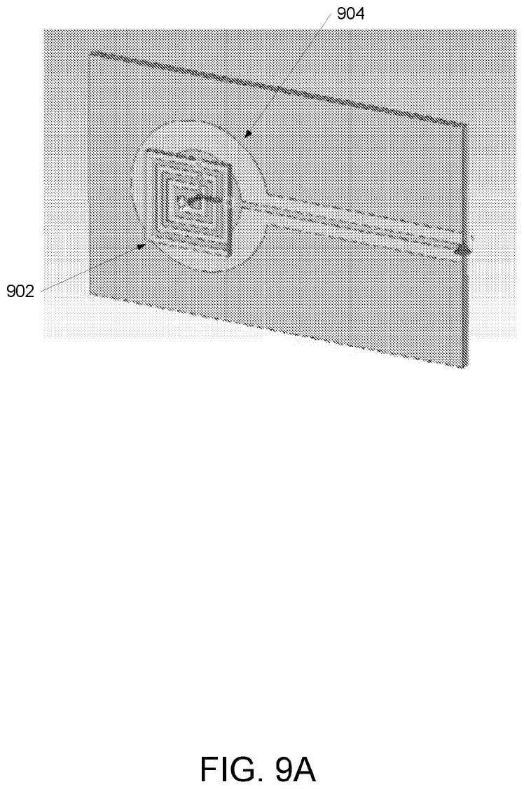

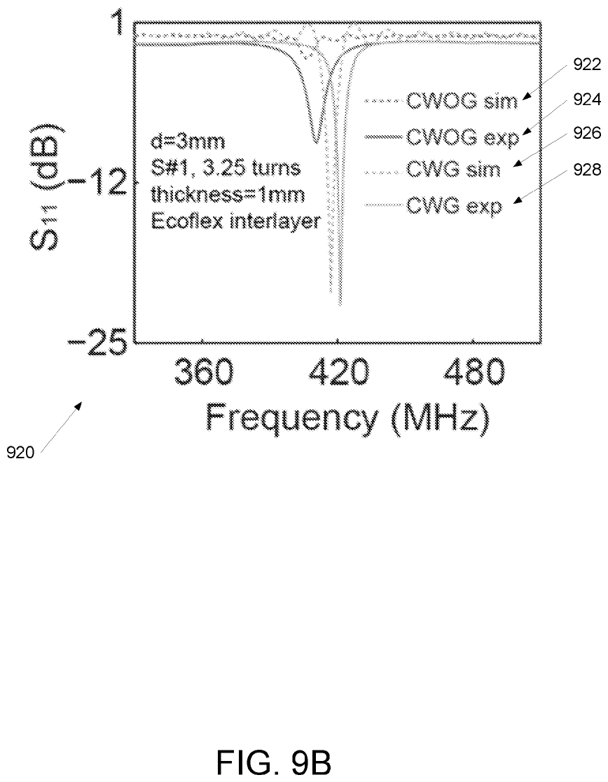

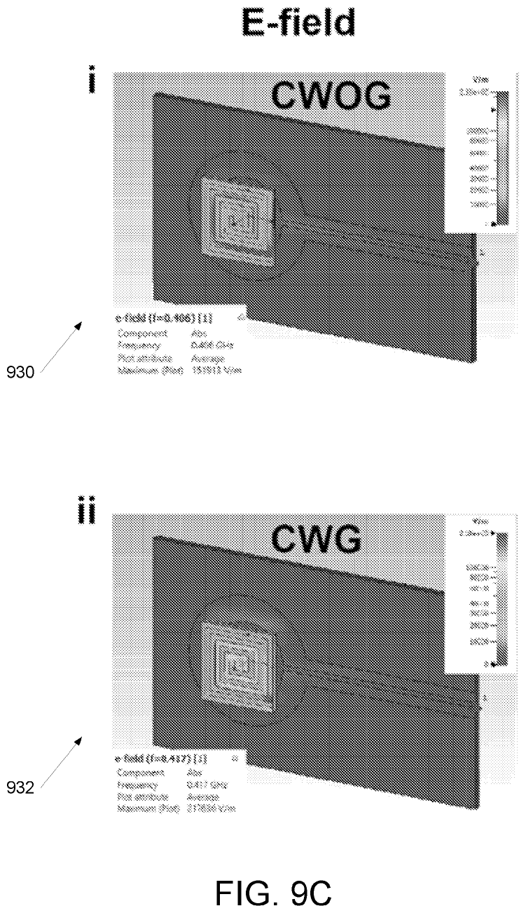

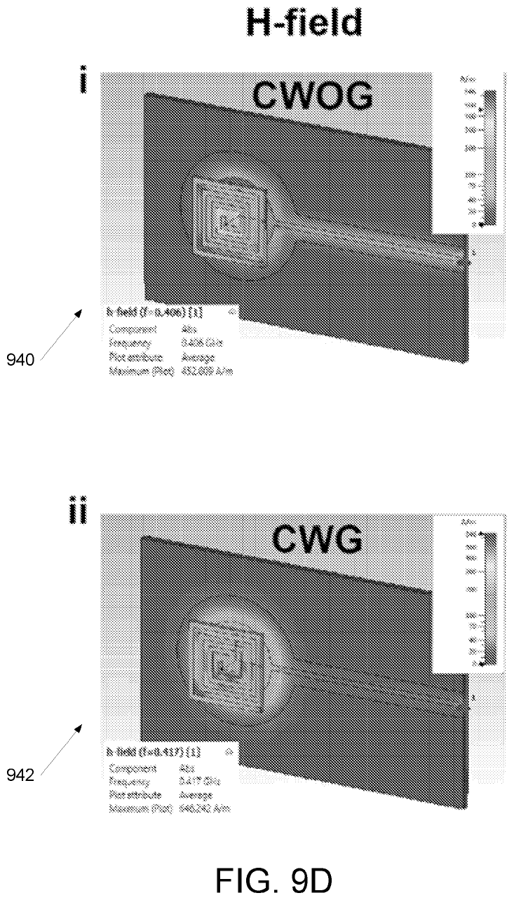

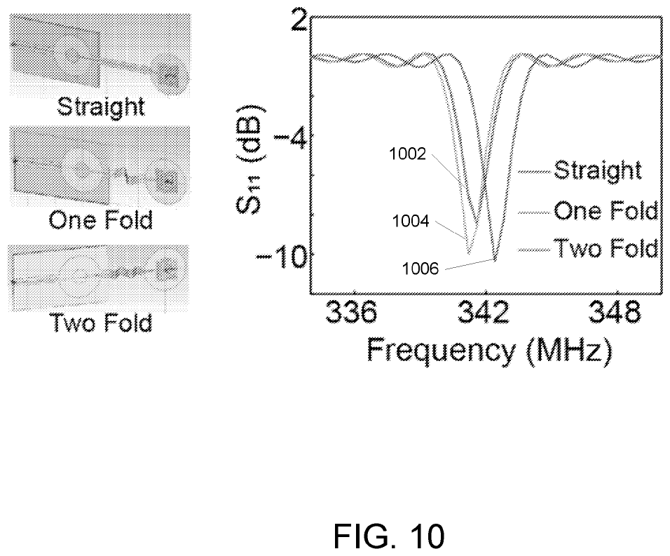

In reference to FIG. 1E, FIG. 1E compares the spectral readout of single and multiple sensors when probed by various readout antennas, with and without an IR interceded within the structure. The present embodiments detail the effect of different vertical distances between the antenna and sensors, which modulates the spectral response due to changing coupling coefficient (see FIG. 8, which illustrates effect of vertical distance between the sensor and the readout coil. The sensor was placed on a 3D printed box (PLA material) and the box was clamped in two supports that moves vertically to change the distance. Change in the vertical distance modulates the coupling between the sensor and the readout coil, which in turn modulates the signal resonant frequency and the magnitude. Change in magnitude is higher in the CWOG 802 (showing results for distance of 0.5 mm 801, 4 mm 803, 7 mm 805, and 10 mm 807), while change in resonant frequency is higher in CWG 810 (showing results for distance of 0.5 mm 811, 4 mm 813, 7 mm 815, and 10 mm 817) and PWG 820 (showing results for distance of 0.5 mm 821, 4 mm 823, 7 mm 825, and 10 mm 827)). FTDT simulation was additionally performed to model the behavior of the sensor and readout coil resonant spectra, and to map the EM field distribution (see FIG. 9, where FIG. 9A illustrates geometry of a sensor 902 and readout coil 904 used in the simulation; FIG. 9B illustrates spectral response 920 of a sensor by simulation (dash line) 922, 926, and by experiment (solid line) 924, 928; FIG. 9C illustrates E-field distributions 930, 932 of a sensor due to CWOG and CWG readout coils, respectively; FIG. 9D illustrates H-field distributions 940, 942 of a sensor due to CWOG and CWG readout coils, respectively. Both E and H fileds are higher in magnitude at their resonance frequency for CWG than CWOG, which yields a higher Q in the S₁₁ spectral response). It can be seen that the grounded structures exhibit a larger EM field close to the readout coil, however this decays more rapidly than the ungrounded structure as we move away from the coil. Both E and H fields are higher with CWG than CWOG at 3 mm separation between the readout coil and sensor—this matches the higher Q measured with CWG. In addition, the present embodiments simulated the effect of bending on sensor readout (see FIG. 10, which illustrates effect of bends in the IR (showing results for straight 1002, one fold 1004, and two folds 1006) on the measured spectral response of the sensor. Parameters for simulation: IR-antenna distance 0.5 mm, IR-sensor distance 1.1 mm, and IR length 110 mm). As shown in the following figure, the impact of 1 or 2 large folds 1004, 1006, respectively, is a minor shift in the measured resonant frequency/magnitude of the sensor. This shift is around +−0.7 MHZ (0.2% shift) in frequency, and 2 dB in magnitude. This puts a limitation on the sensitivity of the sensors in the case of dynamic bending environments, which must possess a sensitivity higher than this “noise” in order to be measured properly.

In further reference to FIG. 1E, the top graph 196 includes CWOG 101, CWG 103, and PWG 105 results, the middle graph 198 includes CWOG 111, CWG 113, and PWG 115 results, and the bottom graph 199 includes CWOG 121, CWG 123, and PWG 125 results. In addition, the middle graph 198 shows the co-readout of three sensors each tuned to different resonant frequencies. Interestingly, the CWOG coil structure exhibits a higher amplitude than the grounded structures in the presence of an IR (see FIG. 1E, bottom graph 199). The slower decay of EM field away from the ungrounded structure improves signal transmission through this intermediate structure, which must be wirelessly coupled to over a set distance. This knowledge can be utilized to optimize network readout and design depending on the presence of an IR, and the coupling distance of the various elements of the network. This will be seen in the measurement/implementation of wireless wristbands and “smart” cups, as further described below. As an additional note, in all such scenarios, the EM field is seen to be strongly confined between individual sensor and the readout coil, which means there is negligible magnetic cross coupling among nearby sensors.

Although specific networks are discussed above with respect to FIGS. 1A-E, any of a variety of networks, as appropriate to the requirements of a specific application, can be utilized in accordance with embodiments of the invention. Various network orientations in accordance with embodiments of the invention are further discussed below.

### Exporation of Various Network Orientations

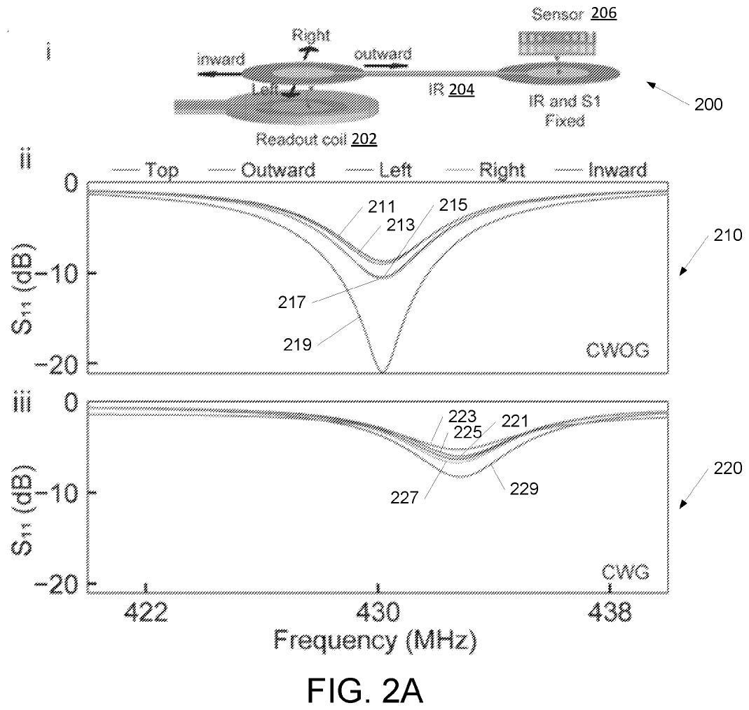

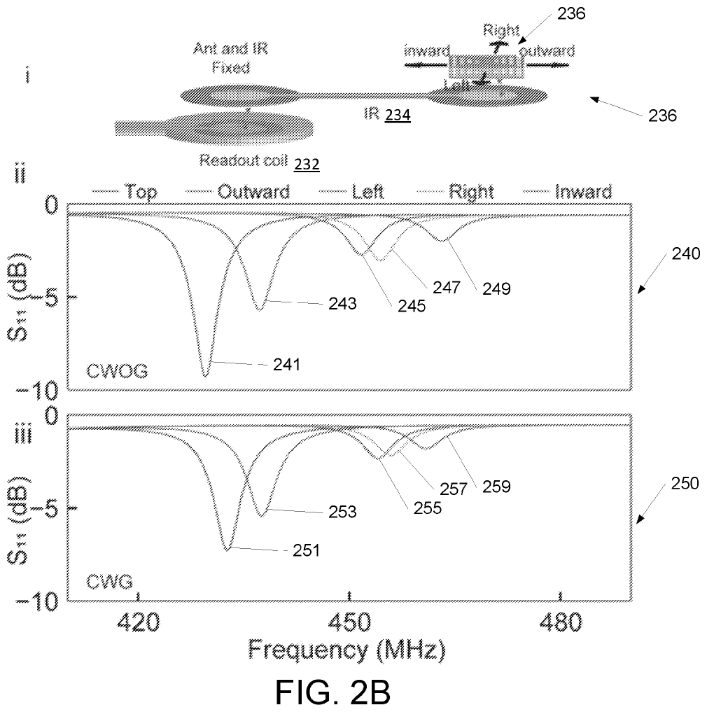

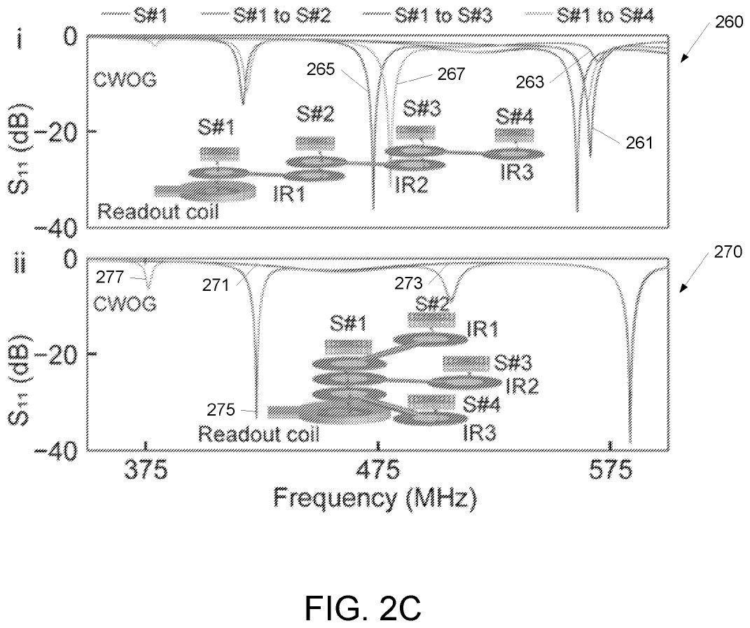

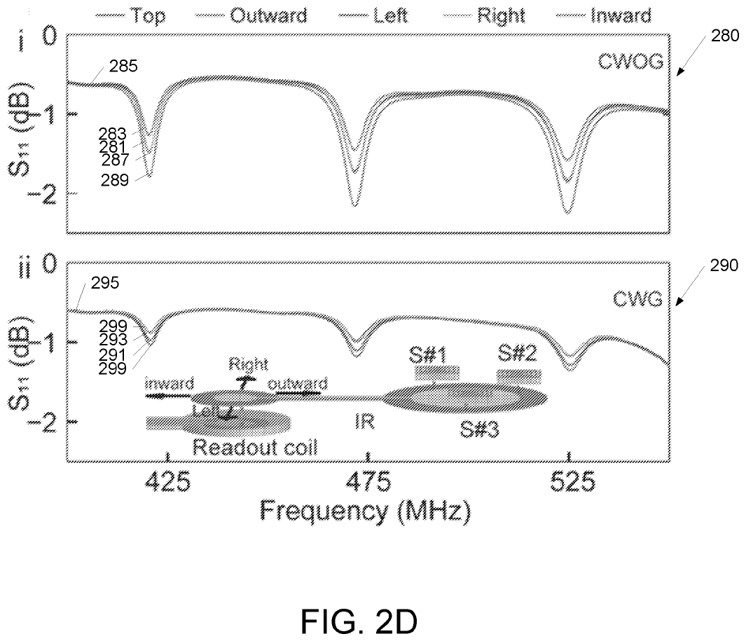

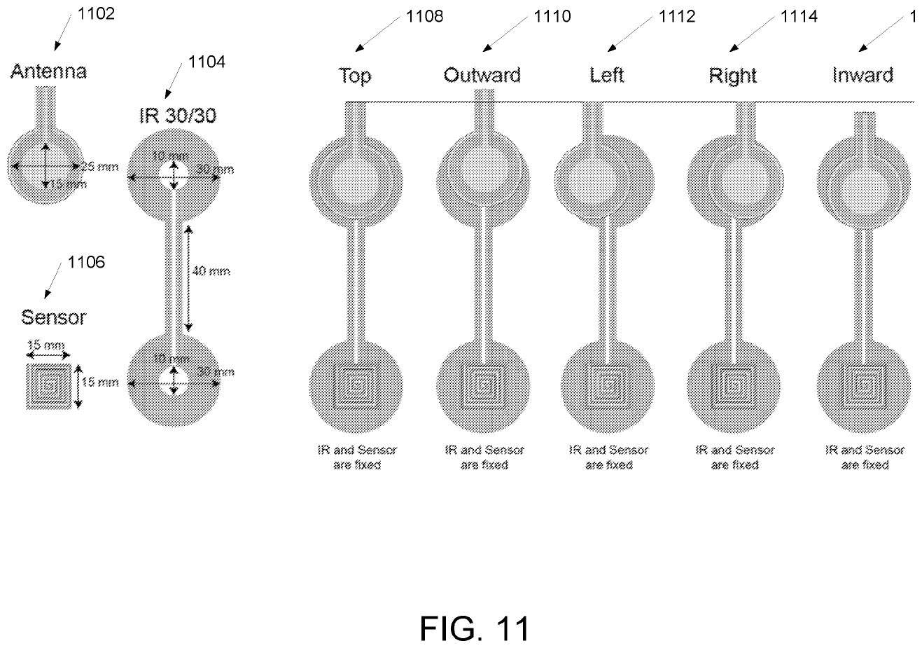

Various network orientations involving the presence of the IR may be explored. The effects of alignment between a readout antenna and IR when IR and sensor orientation is fixed in accordance with an embodiment of the invention is shown in FIG. 2A. FIG. 2A includes (i) schematic presentation 200 of the orientation of the antenna having a readout coil 202, IR 204 and sensor 206, network S₁₁ response by (ii) CWOG 210, and (iii) CWG 220. In referece to FIG. 2A, the effect of the alignment of the readout antenna and an IR30/30 (30 mm loop diameter for both readout coil and sensor coupling) while the IR 204 and sensor 206 placement is fixed was explored (see FIG. 2A, exploded view of the schematic in FIG. 11, which illustrates alignment between IR 1104 and antenna 1102 while sensor 1106 and IR 1104 placement is fixed. The width of the traces in the IR is larger than the width of the traces in the readout coil. This allows misalignment to be within the trace area of the IR-top 1108, outward 1110, left 1112, right 1114, inward 116, shown). As illustrated, the translational alignment between antenna and the IR 204 has little to no effect on the resonant frequency. This stability in the spectral response importantly means that sensors (e.g., sensor 206) that exhibit shifts in resonant frequency due to environmental perturbations remain measurable even if the readout coil 202 is misaligned from the sensor network. This enhanced mechanical stability may be important because this readout coil to network alignment is often not fixed because the reader is commonly brought up to the network and subsequently removed after readout. Note that sensors that shift in magnitude are still measurable given their sensitivity is larger than the magnitude shifts induced by perturbation (this can be tuned by targeting less-sensitive regions to align/re-align the readout coil to the network). In reference to FIG. 2A, the CWOG 210 graph includes results for alignments of top 211, outward 213, left 215, right 217, and inward 219. Further, the CWG 220 graph also includes results for alignments of top 221, outward 223, left 225, right 227, and inward 229.

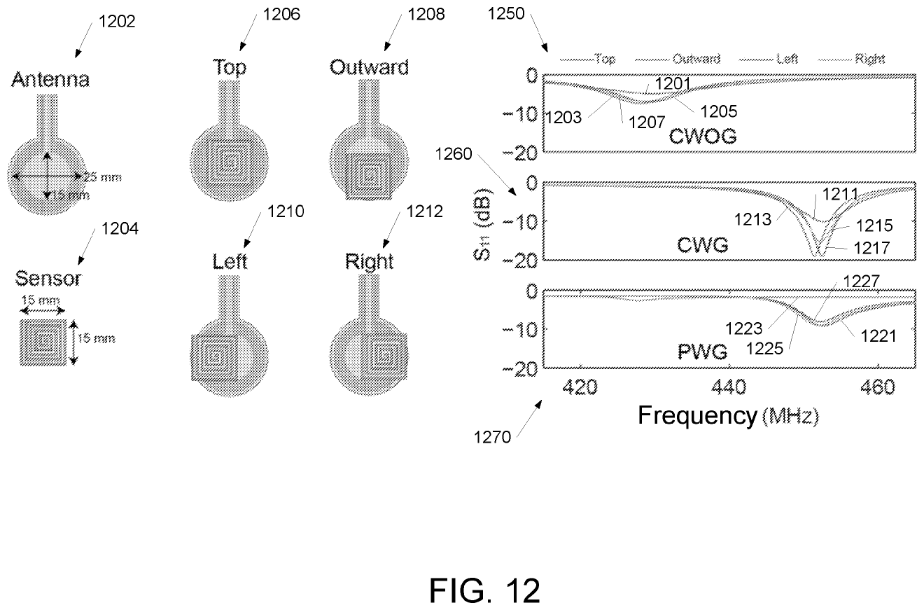

Effects of alignment between IR and sensor while antenna and IR are fixed in accordance with an embodiment of the invention is shown in FIG. 2B. FIG. 2B includes (i) schematic 230 of network orientation, network S₁₁ response by (ii) CWOG 240, and (iii) CWG 250. In reference to FIG. 2B, we fixed the antenna 232 and IR30/30 234 placement, and studied the effect of the IR 232 and sensor 236 alignment. As expected, it was observed that sensor coupling is strongly dependent on the orientation of the sensor 236 with the IR 234, which may result in shifts to both the resonant frequency and the signal amplitude. This means that this IR-sensor alignment should remain fixed throughout measurement. We note that this is the primary purpose of the IR 234, which is flexible/conformable and may permanently route signal to desired regions as required by a specific application. This instability is similar to when the readout coil and sensors exhibit mechanical translations without the presence of an IR (see FIG. 12, which illustrates effects of alignment between antenna 1202 having a readout coil and the sensor 1204 without IR. The measured resonant frequency modulates with changing alignment—top 1206, outward 1208, left 1210, and right 1212 shown. In reference to FIG. 12, the CWOG 1250 graph includes results for alignments of top 1201, outward 1203, left 1205, and right 1207. Further, the CWG 1260 graph also includes includes results for alignments of top 1211, outward 1213, left 1215, and right 1217. Furthermore, the PWG 1270 graph includes results for alignments of top 1221, outward 1223, left 1225, and right 1227). In reference to FIG. 2B, the CWOG 240 graph includes results for alignments of top 241, outward 243, left 245, right 247, and inward 249. Further, the CWG 250 graph also includes results for alignments of top 251, outward 253, left 255, right 257, and inward 259.

Readout of multiple sensors through multiple IR in accordance with an embodiment of the invention is shown in FIG. 2C. FIG. 2C includes Network Si response during (i) series 260, and (ii) parallel extension 270. Insets are the network orientation. In reference to FIG. 2C, multiple IR may additionally be coupled to the readout coil via series or parallel extension, where differing number of sensors are added to the network. In series extension 260, sensors placed further in the network modulate the spectral response due to sensor cross-coupling. This disappears for parallel extension 270, where additional sensors may be added without modulating the measured resonant frequency of previous sensors (in this scenario, sensors are coupled only to the IR but not each other). In reference to FIG. 2C, the series extension 260 graph includes results for S#1 261, S#1 to S#2 263, S#1 to S#3 265, and S#1 to S#6 267. Further, the parallel extension 270 graph also includes results for S#1 271, S#1 to S#2 273, S#1 to S#3 275, and S#1 to S#6 277.

Readout of multiple sensors by IR in accordance with an embodiment of the invention is shown in FIG. 2D. FIG. 2D illustrates the effect of the alignment between readout antenna and IR when IR and sensor orientations are fixed: Network S₁₁ response by (i) CWOG 280, (ii) CWG 290. Inset is the network orientation. In reference to FIG. 2D, multi-sensor networks are additionally stable to mechanical translation. These results broadly indicate that IR interceded sensor networks may provide stabilized readout given a wide variety of scenarios. One limitation of using the IR may be elongating the ends will lead to a reduction in signal amplitude (and thus limits the practical sensitivity of the measurement). There may be a direct trade-off, where very long distances will require either higher-sensitivity sensors, or higher input power in order to resolve measurements.

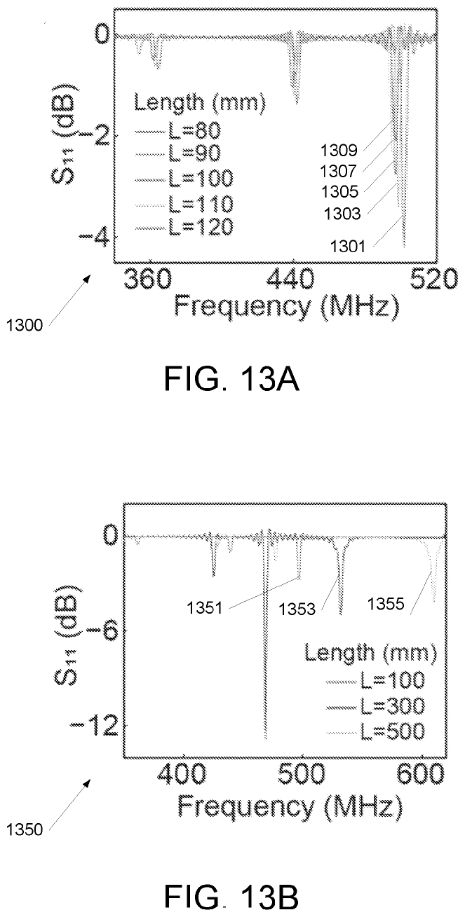

Additional simulations were performed to illustrate the effect of increased length on the measured S₁₁ (see FIG. 13, which illustrate length study: a) effect of small changes in the length 1300 (showing results for length=80 mm 1301, 90 mm 1303, 100 mm 1305, 110 mm 1307, and 120 mm 1309), b) large changes in the length 1350 (showing results for length=100 mm 1351, 300 mm 1353, and 500 mm 1355). Parameters for simulation: IR-antenna distance 0.5 mm, and IR-sensor distance 1.1 mm). At 50 cm the measured S₁₁ of sensors does decrease in comparison to shorter distances, as various sensors will exhibit magnitude shifts of 1 to 4 dB at this distance (higher frequency sensors are more robust to increased distances). From these findings it appears as though at very long distances sensors may be pushed to higher operating frequencies to maintain similar readout sensitivity. In general, we have found even −1 dB of amplitude response to be sufficient for proper measurement of various RF sensors. In reference to FIG. 2D, the CWOG 280 graph includes results for alignments of top 281, outward 283, left 285, right 287, and inward 289. Further, the CWG 290 graph also includes results for alignments of top 291, outward 293, left 295, right 297, and inward 299.

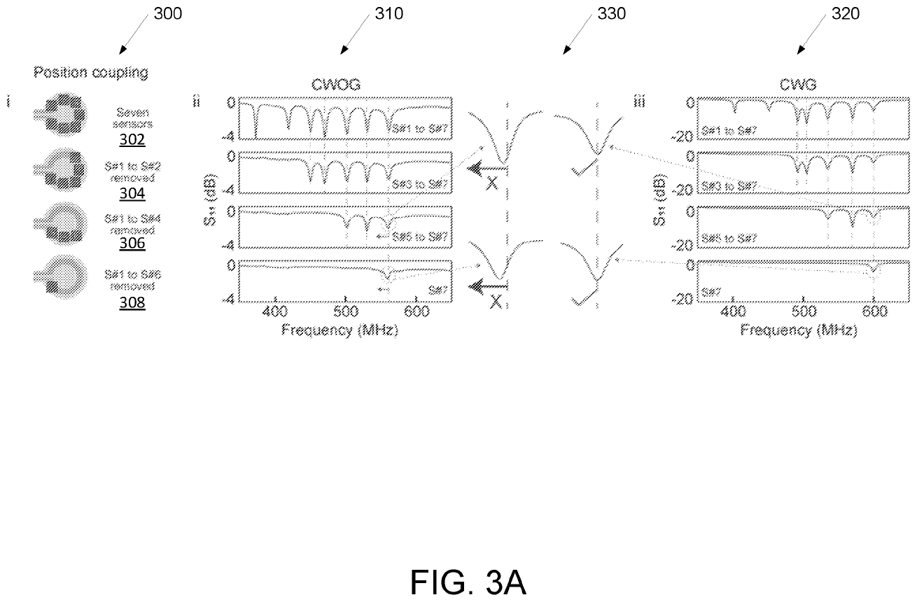

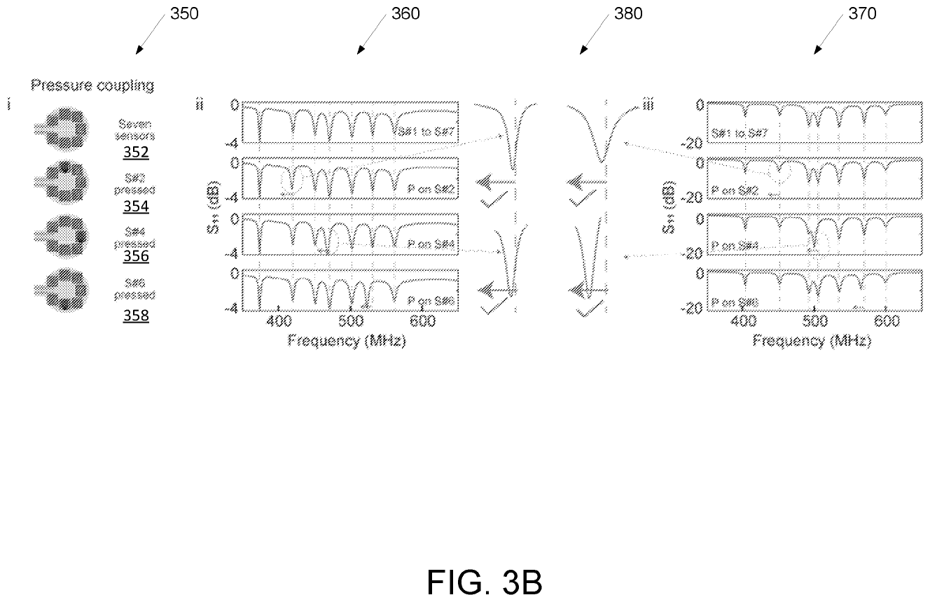

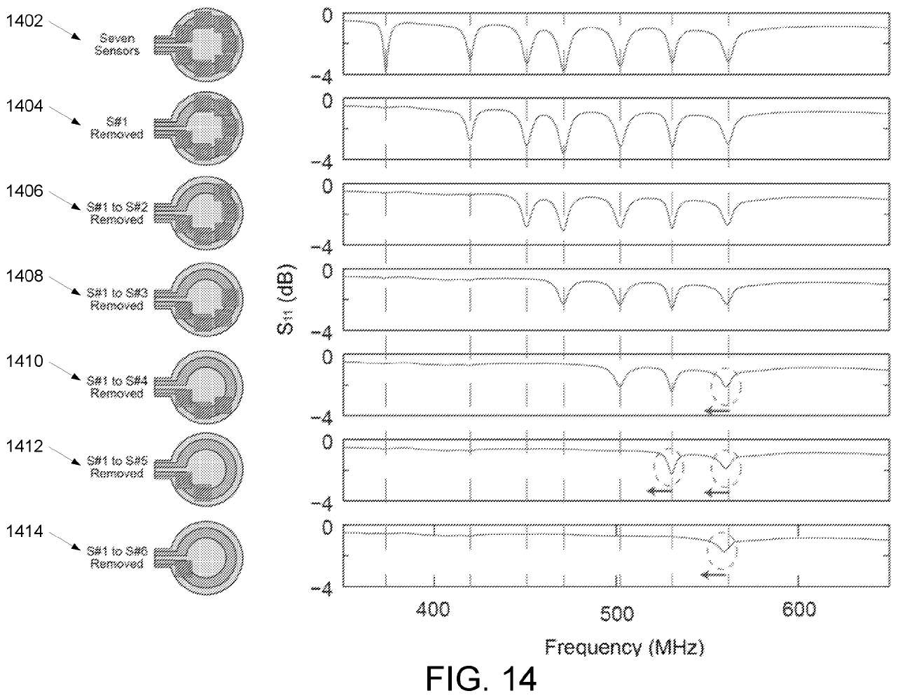

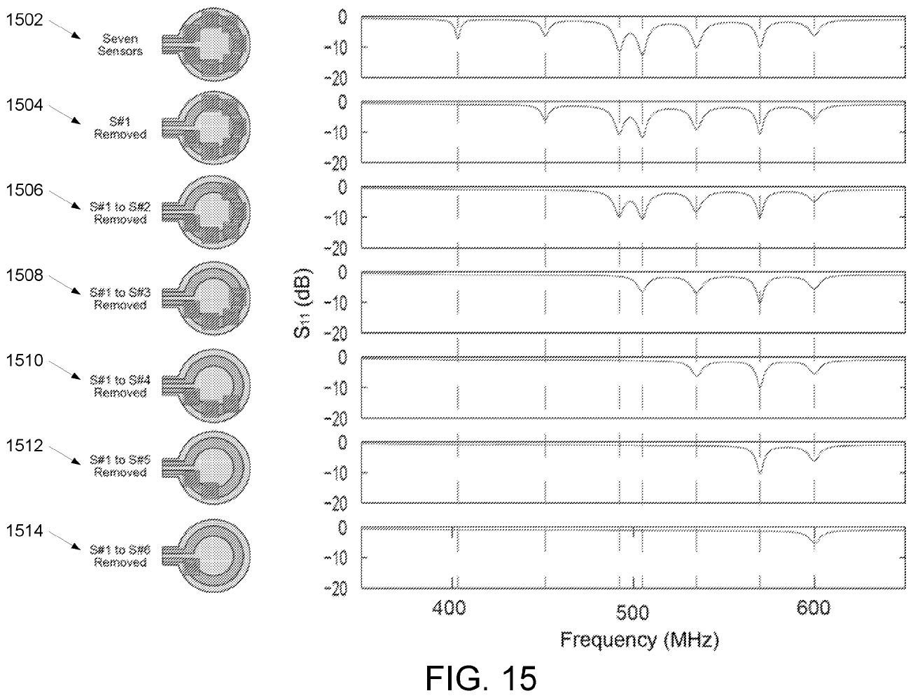

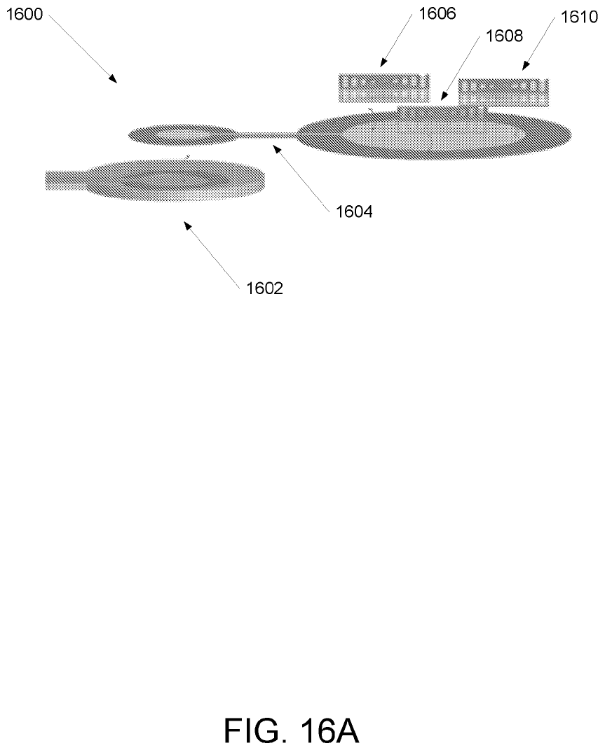

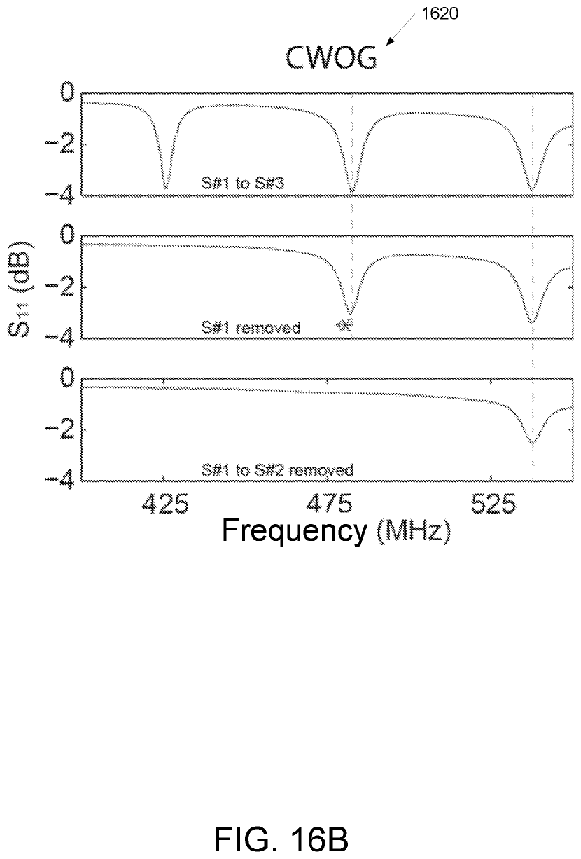

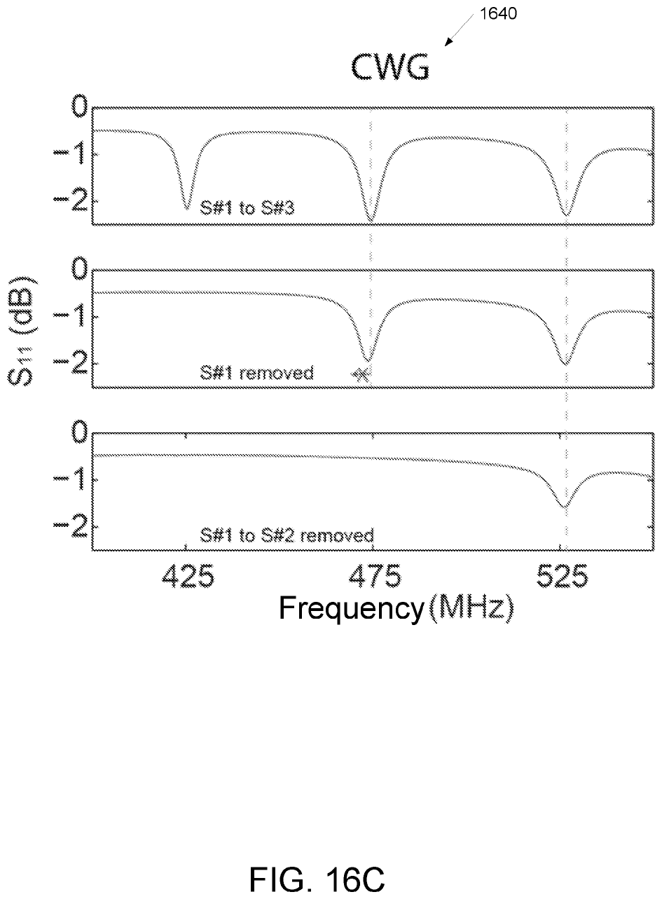

In such a passive network, a large number of sensors may potentially be accommodated, the limits of which may be assessed through measurement of the cross-coupling among sensors. Effect of positional coupling between sensors in accordance with an embodiment of the invention is shown in FIG. 3A. FIG. 3A includes (i) schematic presentation of sensor array orientation during experiment (e.g., seven sensors 302, S#1 to S#2 removed 304, S#1 to S#4 removed 306, and S#1 to S#6 removed 308), and S₁₁ response from sensors with (ii) CWOG 310 and (iii) CWG 312 readout antennas. In between is a blow-up plot 330 highlighting the shift or lack of shift in resonant frequency due to coupling. In reference to FIG. 3A, we initially tested sensor positional coupling as they were arrayed in increasing numbers above readout coils. This type of coupling may not be as important as sensor measurement coupling, the results of which will follow. Seven sensors 302 were placed initially on the readout coil (the minimum physical distance of the adjacent sensors are 2 mm) and sensors were removed one by one 304, 306, 308. It was found that for ungrounded readout coils, removal of sensors from a dense network could lead to small shifts in the measured resonant frequency of remaining sensors on the network (see FIG. 3A(ii) 310). On the other hand, for CWG, there is no effect due to removal of sensors from seven to one shown in FIG. 3A(iii) 320 (full data on these experiments is shown in FIG. 14 for CWOG and in FIG. 15 for CWG. FIG. 14 illustrates positional coupling effect among sensors measured by one-by-one removal above a CWOG antenna (e.g., seven sensors 1402, S#1 removed 1404, S#1 to S#2 removed 1406, S#1 to S#3 removed 1408, S#1 to S#4 removed 1410, S#1 to S#5 removed 1412, and S#1 to S#6 removed 1414). FIG. 15 illustrates positional coupling effect among sensors measured by one-by-one removal above a CWG antenna (e.g., seven sensors 1502, S#1 removed 1504, S#1 to S#2 removed 1506, S#1 to S#3 removed 1508, S#1 to S#4 removed 1510, S#1 to S#5 removed 1512, and S#1 to S#6 removed 1514)). In addition, as described and shwon above, the measured sensor amplitude is stronger for CWG during this direct multi-sensor readout. This type of coupling effect due to sensor placement was additionally tested with an IR (see FIG. 16, which illustrates position coupling effect among sensors with an IR30/50: (a) Schematic 1600 of the readout coil 1602, IR 1604, and sensors 1606, 1608, 1610, alongside network spectral response with (b) CWOG 1620 or (c) CWG 1640. Both CWOG 1620 and CWG 1640 show cross coupling among sensors), and shows a positional effect for both CWOG and CWG in agreement with this observed effect. This implies that given a dynamic sensor network wherein sensors may be picked-and-placed, grounded RF elements will simplify sensor measurement due to minimal positional coupling. One fundamental limitation of such system may be the maximum number of sensors that can be measured. For low-cost (wearable) VNA systems that accurately measure response up to ˜1.5 GHZ, the primary limitation may come in the bandwidth that sensors occupy. Generally, approximately 100 MHz band per sensor may be more than sufficient to properly assay individual sensors (smaller bandwidth may be required for more sensors that shift less in frequency with perturbation). For low-cost systems, with sensors that occupy 100 MHz, it may be assumed that a network may accommodate around 15 RF sensors.

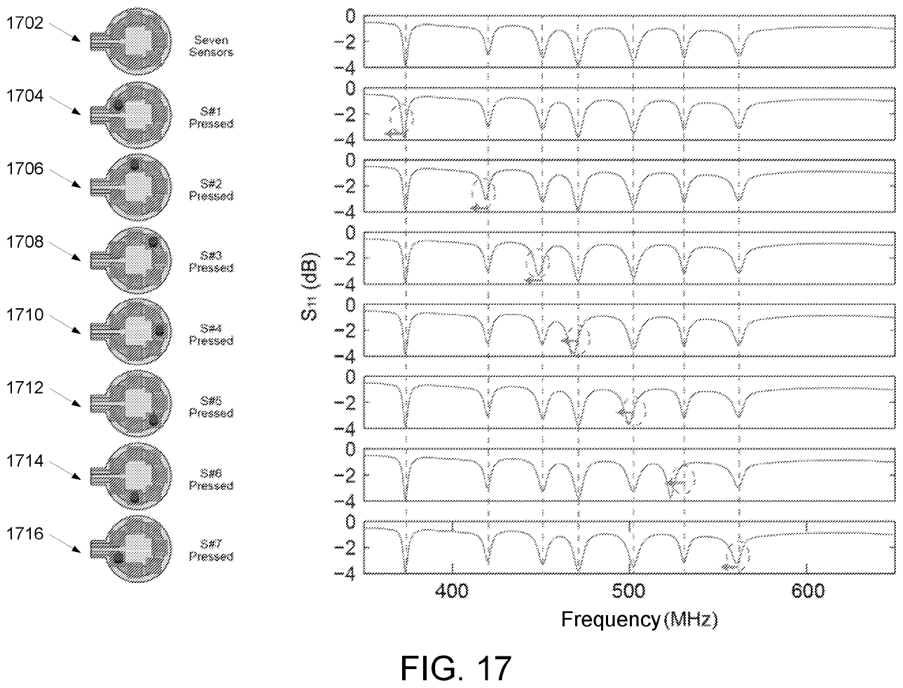

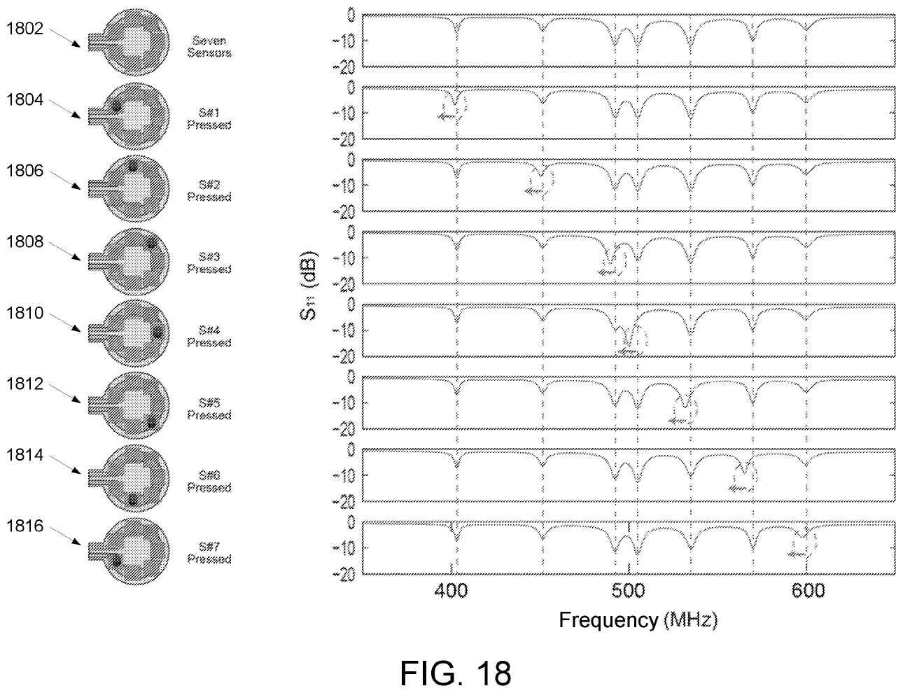

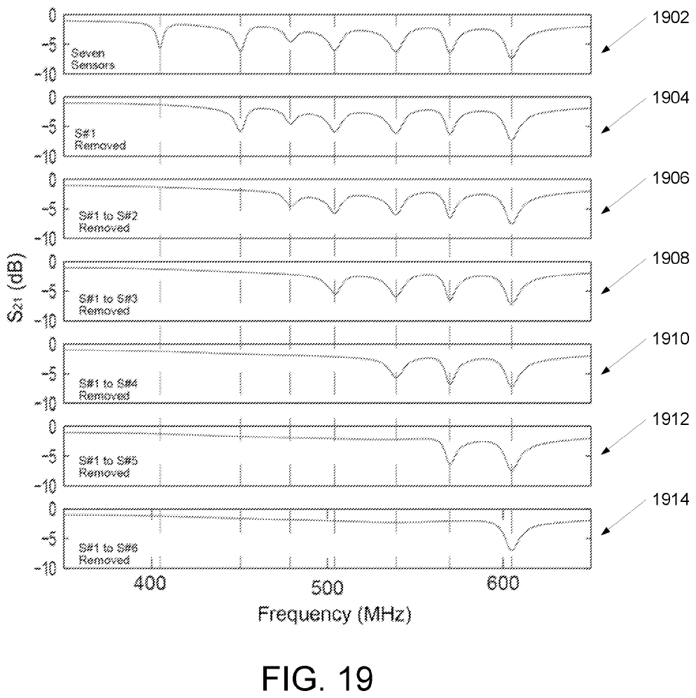

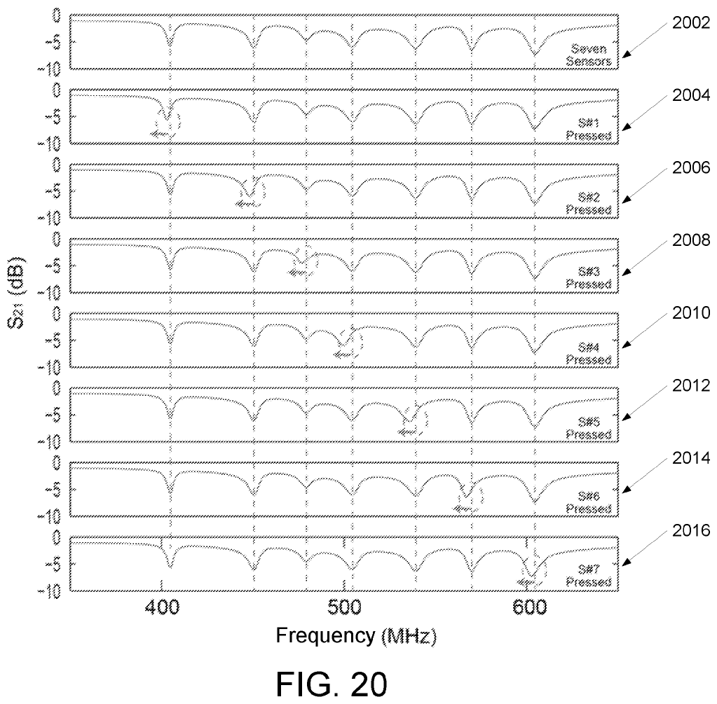

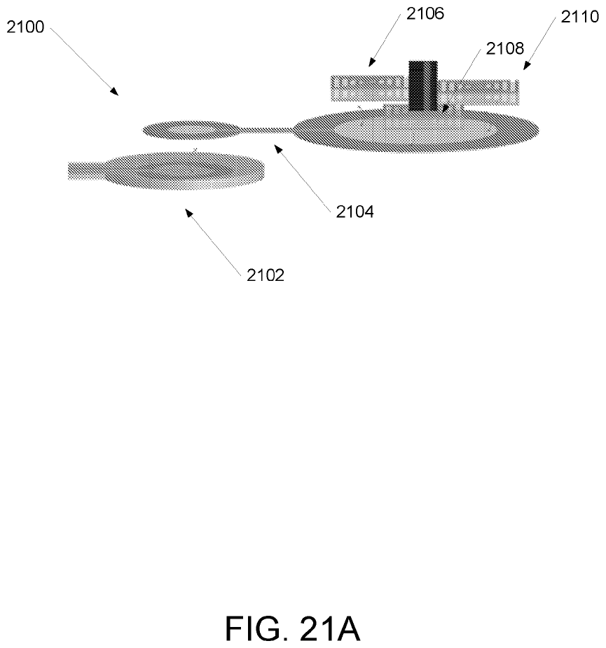

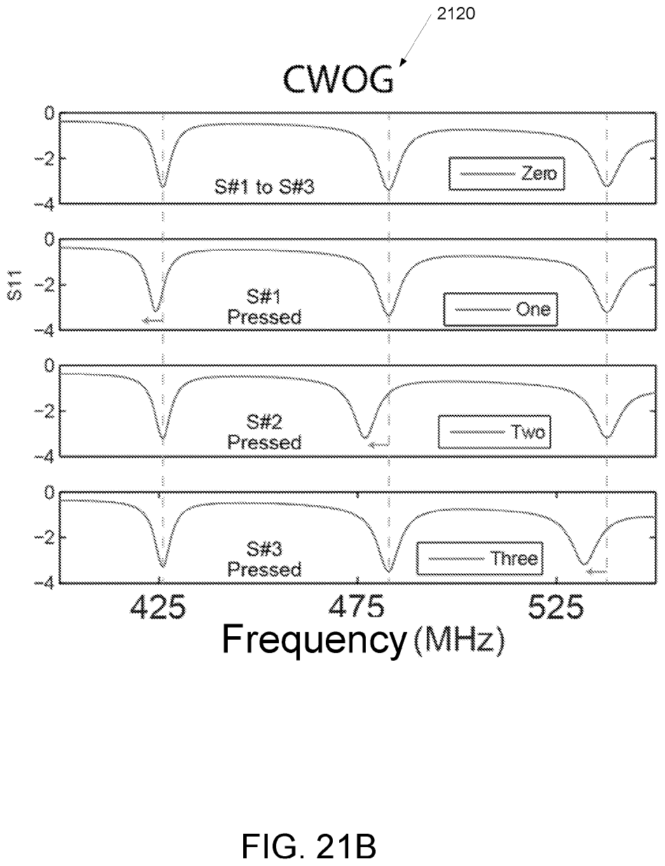

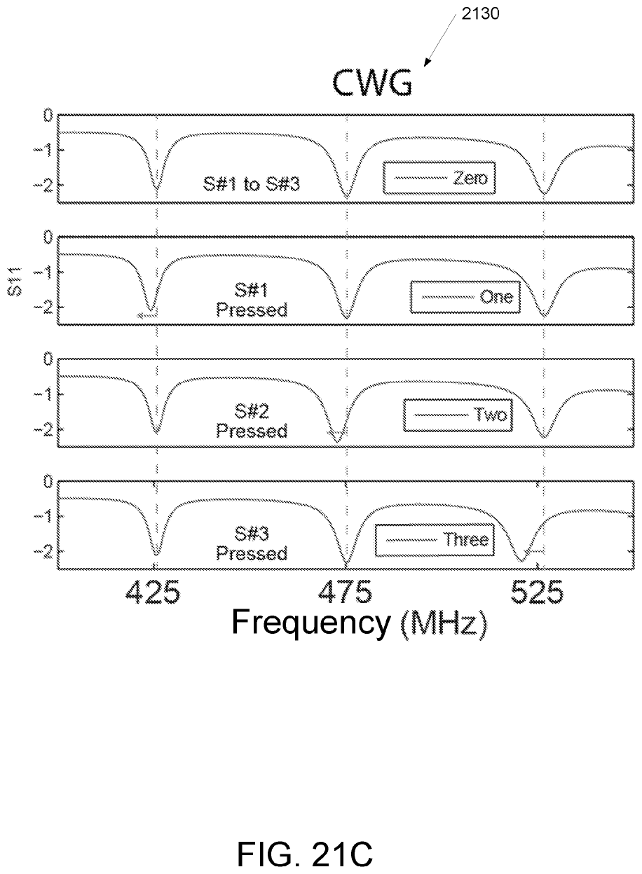

More important than the above, may be assessing how the perturbation of single RF sensor may impact the total spectral response of the network. Effect of sensimetric coupling between sensors (applied pressure) in accordance with an embodiment of the invention is shown in FIG. 3B. FIG. 3B includes (i) schematic presentation 350 of applying stimuli to various sensors, and S₁₁ response from sensors with (ii) CWOG 360 and (iii) CWG 370 readout antennas. In between is a blow-up plot 380 highlighting the shift in resonant frequency of the spectral peak linked to respective sensors. No other peak exhibits a shift. In reference to FIG. 3B, seven pressure sensors 352 were placed on the readout coil and each sensor perturbed in sequence (e.g., S#2 pressed 354, S#4 pressed 356, and S#6 pressed 358), (see FIG. 17 for CWOG and FIG. 18 for CWG. FIG. 17 illustrates coupling among sensors due to individual sensor perturbation (pressure) in CWOG (e.g., seven sensors 1702, S#1 pressed 1704, S#2 pressed 1706, S#3 pressed 1708, S#4 pressed 1710, S#5 pressed 1712, S#6 pressed 1714, and S#7 pressed 1716). FIG. 18 illustrates coupling among sensors due to individual sensor perturbation (pressure) in CWOG (e.g., seven sensors 1802, S#1 pressed 1804, S#2 pressed 1806, S#3 pressed 1808, S#4 pressed 1810, S#5 pressed 1812, S#6 pressed 1814, and S#7 pressed 1816).). In these mechanical sensors, the resonant frequency will shift due to an applied mechanical pressure. For such a static network, for CWOG, CWG, and PWG, no disturbance/cross coupling in the total spectra of the network due to the perturbation in individual sensors was found (see FIG. 3B(ii) 360 and 3B(iii) 370, PWG shown in FIGS. 19 and 20. FIG. 19 illustrates positional coupling effect among sensors measured by one-by-one removal above a PWG antenna (e.g., seven sensors 1902, S#1 removed 1904, S#1 to S#2 removed 1906, S#1 to S#3 removed 1908, S#1 to S#4 removed 1910, S#1 to S#5 removed 1912, and S#1 to S#6 removed 1914). FIG. 20 illustrates coupling among sensors due to individual sensor perturbation (pressure) in PWG (e.g., seven sensors 2002, S#1 pressed 2004, S#2 pressed 2006, S#3 pressed 2008, S#4 pressed 2010, S#5 pressed 2012, S#6 pressed 2014, and S#7 pressed 2016)). Additionally, no sensimetric coupling is observed with an additional interceding IR element (see FIG. 21, which illustrates coupling among sensors due to individual sensor perturbation (pressure) with an interceding IR: (a) schematic 2100 of the placement of the readout coil 2102, IR 2104, and sensors 2106, 2108, 2110, and network spectral response for (b) CWOG 2120, or (c) CWG 2130. The network exhibits no sensimetric coupling as long as the number of sensors stays static). In conjunction with measurements on sensor positional coupling, this data suggests that the presence of individual sensors may modulate the induced EM field around the readout coil with ungrounded readout (thus perturbing measurements if sensors are removed), however individual sensor response does not directly cross-couple to the total network. Importantly, this implies that with a static and defined network, given any measurement modality used or with/without the presence of an IR, individual sensor response links exclusively to its designated wavelength. As further described below, such static networks may readily be engineered by embedding coils and sensors along structures with our flexible fabrication protocols.

Although specific network orientations are discussed above with respect to FIGS. 2A-3B, any of a variety of networks, as appropriate to the requirements of a specific application, can be utilized in accordance with embodiments of the invention. Implementation of networks in accordance with embodiments of the invention are further discussed below.

### Implementation of Programmable Multi-Wavelength RF Spectrometry Networks

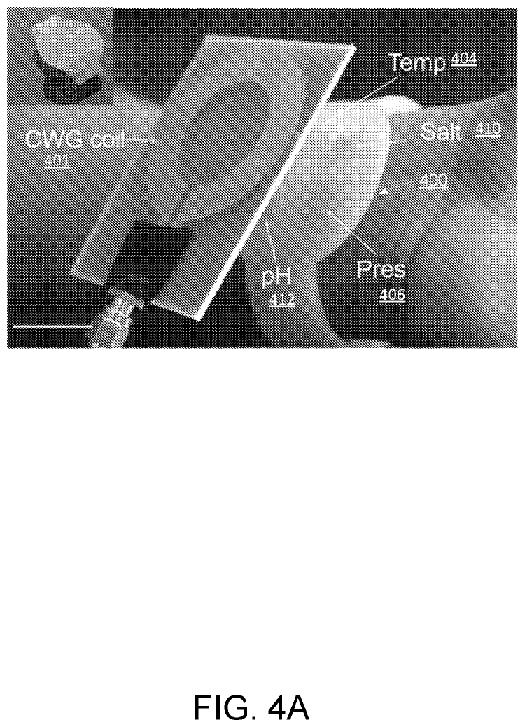

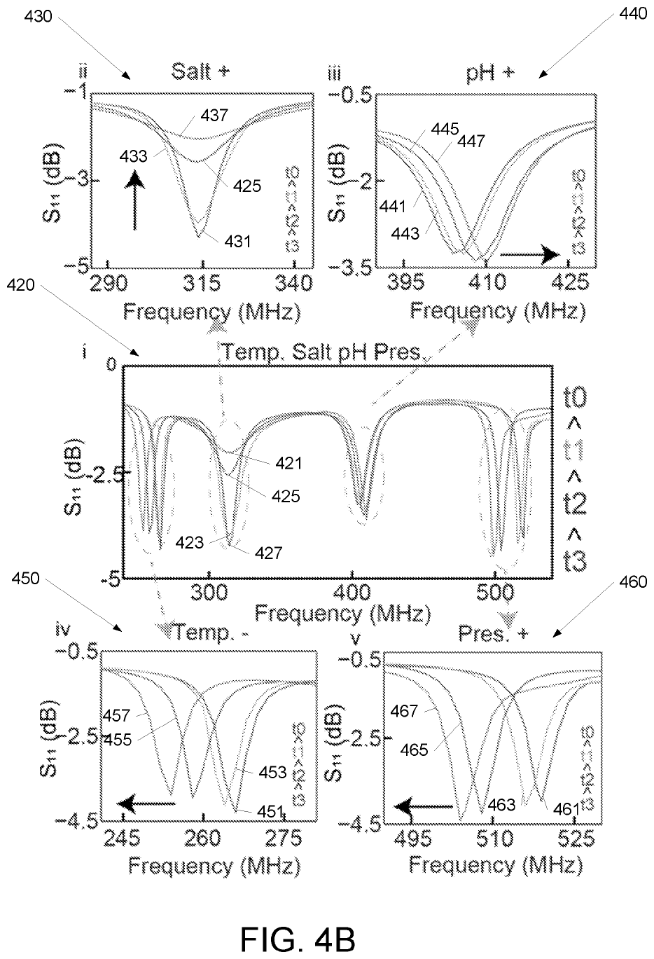

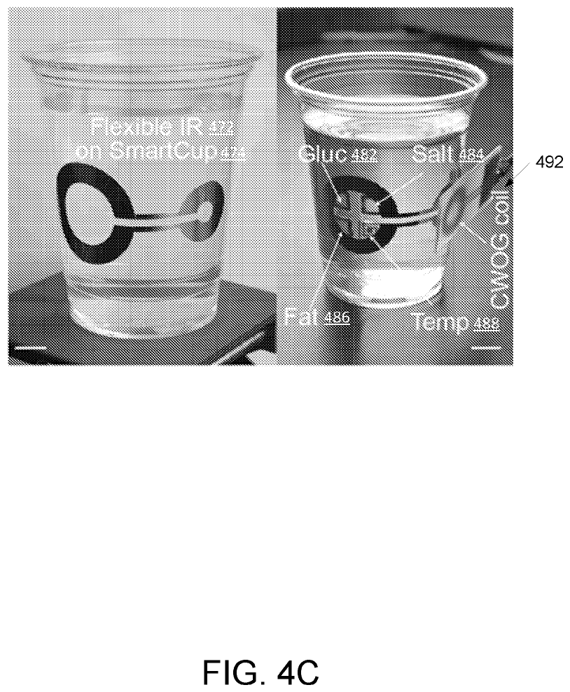

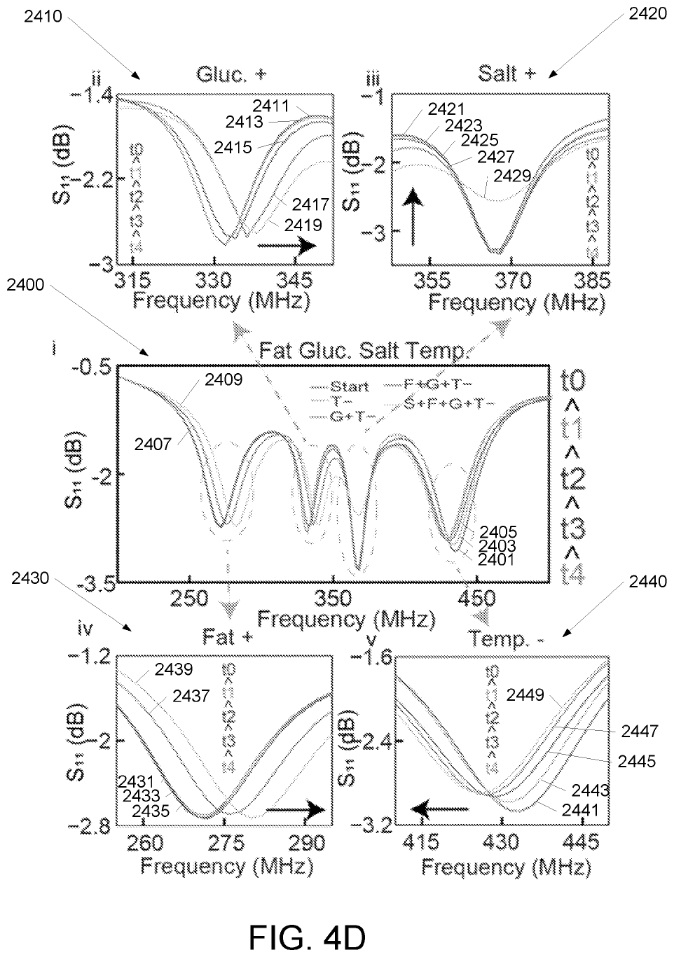

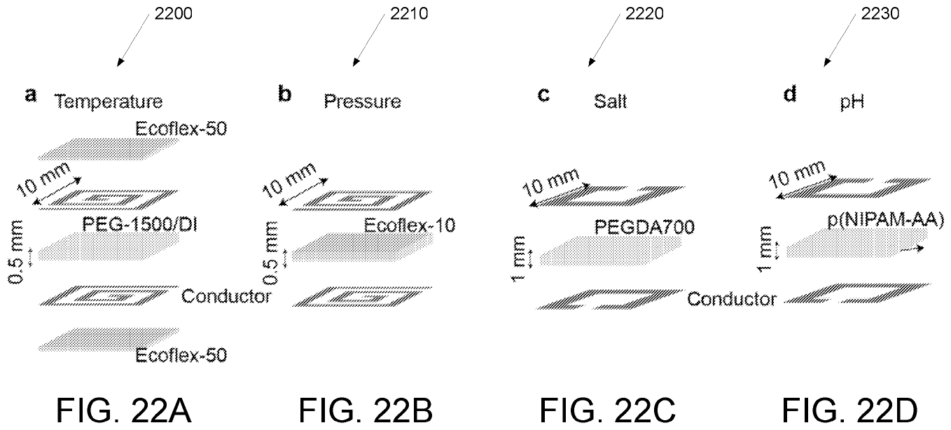

Passive wireless networks may be utilized to monitor the chemophysical state of objects and environments relevant to daily life. Exemplary studies are further described below. First was with a wristband with four sensors that enable co-readout of salt, pH, temperature and pressure simultaneously (sensor structures are shown in FIG. 22, which illustrates sensors used in wristband: (a) PEG-1500 interlayer temperature sensor 2200, (b) Ecoflex-10 interlayer pressure sensor 2210, (c) PEGDA700 hydrogel interlayer salt sensor 2220, and (d) p (NIPAM-AA) hydrogel interlayer pH sensor 2230). In general, capacitive-based sensors may shift up to 20% in resonant frequency with varying input, while loss-based sensors will modulate up to 80% in magnitude. Multiparametric readout (e.g., using a CWG coil 401) from a wearable wristband 402 in accordance with an embodiment of the invention is shown in FIG. 4A. In reference to FIG. 4A, a wristband 402 with temperature and pressure sensors 404, 406, respectively, may be completely sealed within a silicone 408, however salt and pH sensors 410, 412, respectively, may have a bottom side opening to enable access to the sweat.

As further described above, passive sensors may individually be readout wirelessly without any microelectronics at the sensing node. Spectrometric co-readout of sensor state in accordance with an embodiment of the invention is shown in FIG. 4B. FIG. 4B includes (i) evolution of spectra after completed perturbations 420, and (ii-v) zoom-in of network S₁₁ response due to modulating salt (0 to 10 mg/dL) 430, pH (4 to 7.4) 440, temperature (40° C. to room) 450, and pressure (manual) 460. Such sensors may be co-monitored with an intermediate relay fused on textile (see FIG. 1B), or directly with the readout as is shown in FIG. 4A. As CWG elicits a higher magnitude response, if there is no IR, a 5 cm CWG antenna may be used to co-read sensor response simultaneously through direct readout.

In further reference to FIG. 4B, the ability of sensors to monitor analytical to physical signals around human subjects was tested. Typical probing power for VNA (wearable and otherwise) maxes out at around −6 to −5 dbm (˜300 mW). This is below NFC power standards (˜1 W), which have a measured SAR of over an order of magnitude below upper limits for the human body. It is estimated that the allowed maximum power would be a bit over an order of magnitude greater than our current VNA (around the power limit utilized in most Qi chargers, e.g., 10 W). This type of excess power is typically unnecessary if sensors are well-designed around bodily stimuli. FIG. 4B(i) 420 shows the original recorded spectra and modified spectra, where individual ii) salt 430, iii) pH 440, iv) temperature 450 and v) pressure sensor 460 response is shown a larger view. The stimuli were generated individually as follows (to validate the lack of cross-coupling among sensors): the temperature sensor was heated by hot air flow, pressure sensor was mechanically stimulated by various weights, NaCl was added to the salt sensors, while DI water was added to the pH sensor. As expected, the resonant frequency of the temperature sensor decreases while cooling as the permittivity of the PEG-1500 interlayer material increases at lower temperature. Resonant frequency of the pressure sensor decreases with pressure as pressure decreases the interlayer thickness. The magnitude of the signal of the salt sensor decreases as salt penetrates and increases the conductivity of the interlayer PEGDA700 hydrogel. The resonant frequency of the pH sensor increases with the DI water (pH˜7), as the p (NIPAM-co-AA) swells from pH 4 to pH 7. Such a wearable wristband enables a passive and wireless multiparamatric readout of the bodily state without any electronics required on the body. In reference to FIG. 4B, the completed perturbations 420 graph includes results at time zero (t0) 421, time one (t1) 423, time two (t2) 425, and time three (t3) 427, where to <t1<t2<t3. Further, the salt response 430 graph includes results at t0 431, t1 433, t2 435, and t3 437, where t0<t1<t2<t3. Furthermore, the pH response 440 graph includes results at t0 441, t1 443, t2 445, and t3 447, where to <t1<t2<t3. Moreover, the temperature response 450 graph includes results at t0 451, t1 453, t2 455, and t3 457, where t0<t1<t2<t3. In addition, the pressure response 460 graph includes results at to 461, t1 463, t2 465, and t3 467, where to <t1<t2<t3.

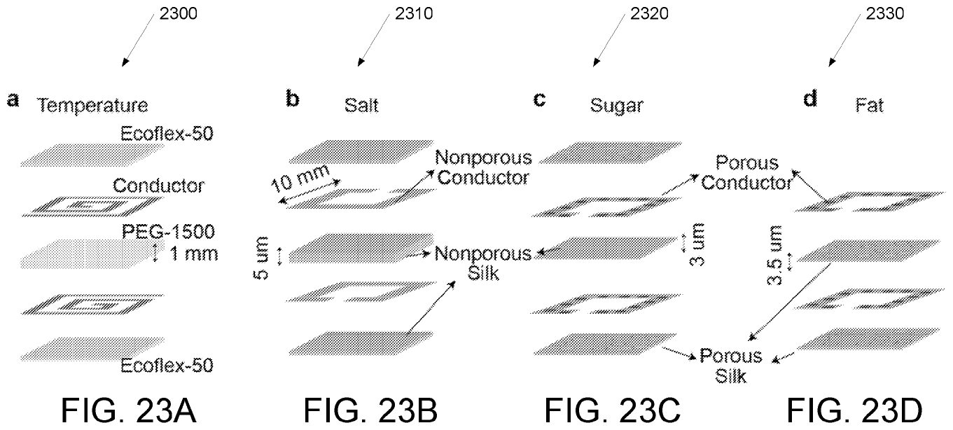

To characterize a functional network containing an IR, we developed a Smartcup that is integrated with novel biosensors (sensor structures shown in FIG. 23, which illustrate sensors in SmartCup: (a) PEG-1500 interlayer temperature sensor 2300, (b) nonporous silk fibroin interlayer salt sensor 2310, (c) nonporous silk fibroin interlayer sugar sensor 2320, and (d) porous silk fibroin interlayer fat sensor 2330) for the discrimination and co-readout of nutrients direct from food. A multi-scale engineering of silk biopolymer-interlayer constructs to synthesize different sensors tuned to directly measure salts, sugars, and fat content from food was utilized. A major advantage of the spectral approach demonstrated herein is that it can measure varying optimized nutrient sensors simultaneously, easing the co-readout of multiple nutrients in complex inputs. In addition, a temperature sensor alongside three optimized nutrient sensors (tuned to salt, sugar, fat) in the inner side of the Smartcup was utilized. These sensors were carefully aligned to an IR that was fixed on the outer side of the smart cup. This forms a stable, passive wireless network with zero-electronics that is affixed on a cup.

A Smartcup for co-monitoring nutrients in a drink in accordance with an embodiment of the invention is shown in FIG. 4C. As CWOG elicits a higher magnitude response from the network if an IR is used, a 2.5 cm CWOG antenna with the IR to co-readout the sensors response simultaneously was utilized. In reference to FIG. 4C, left shows placement of a flexible IR 472 on the Smartcup 474, and right shows placement of the sensor configured to measure glucose 482, salt 484, fat 486, and temperature 488, IR 490 and antenna 492.

Spectrometric co-readout of sensors state in accordance with an embodiment of the invention is shown in FIG. 4D. FIG. 4D includes (i) 2400 evolution of spectra after completed perturbations, and (ii-v) zoomed network S₁₁ response of the sugar-optimized 2410 (0 to 100 g/L), salt-optimized 2420 (0 to 25 mg/dL), fat-optimized 2430 (0 to 20 μL), and temperature 2440 (50° C. to room) sensors after completed perturbations. Scale bars are 2 cm. Testing of the nutrient monitoring from the Smartcup was performed, with reports on temperature, salt, sugar and fat as shown in FIG. 4D. FIG. 4D(i) 2400 is the original recorded signal and modulated response, where ii) glucose 2410, iii) salt 2420, iv) fat 2430 and v) temperature 2440 sensor temporal response is each highlighted in a larger view. These sensors have previously been validated to measure nutrient content while directly exposed to foods (teas, meat, milk, etc.), however they do exhibit sensimetric cross-coupling in nutrient response because they are partially-selective (this is decoupled using post-processing analysis). To properly validate that individual sensors do not cross-couple to the full spectra of network each biosensor may be probed in a mini-well through individual perturbation of their respective target nutrient. In addition, all sensors exhibit a response time which must be monitored. The temperature sensor was heated to 50° C. and cooled in a 40° C. environment, validating the temperature sensor response does not elicit a change in the readout of other sensors. Glucose was then added to the sugar biosensor, and this increases the resonant frequency due to biopolymer swelling. At the same time, the temperature sensor is still modulating to a lower frequency because to residual lag in the temperature sensor response, however, the remaining sensors still do not exhibit any change as they have not undergone perturbation. Next, oleic acid is added to the fat sensor, where replacement of high permittivity water with low permittivity oleic acid reduces the capacitance of the structure. Now, all the sensors except the salt-optimized biosensor may exhibit expected temporal shifts in accordance with the characteristics of the individual sensor. Finally, NaCl was added to the salt sensor, which increase the conductivity of the interlayer silk and reduces the signal Q/magnitude. The complete spectra of the Smartcup stabilizes to its final state in accordance with the final state of each individual temperature or nutrient sensor. This validates the measurement capabilities of flexible/reactive passive networks in a practical setting. In reference to FIG. 4D, the completed perturbations 2400 graph includes results at t0 2401, t1 2403, t2 2405, t3 2407, and time four (t4) 2409 where to <t1<t2<t3<t4. Further, the sugar-optimized 2410 graph includes results at t0 2411, t1 2413, t2 2415, t3 2417, and t4 2419 where t0<t1<t2<t3<t4. Furthermore, the salt-optimized response 2420 graph includes results at t0 2421, t1 2423, t2 2425, t3 2427, and t4 2429, where t0<t1<t2<t3<t4. Moreover, the fat-optimized response 2430 graph includes results at t0 2431, t1 2433, t2 2435, t3 2437, and t4 2439, where to <t1<t2<t3<t4. In addition, the temperature response 2440 graph includes results at t0 2441, t1 2443, t2 2445, t3 2447, and t4 2449 where t0<t1<t2<t3<t4.

Although specific network implementations are discussed above with respect to FIGS. 4A-D, any of a variety of network implementations, as appropriate to the requirements of a specific application, can be utilized in accordance with embodiments of the invention. Networks design considerations in accordance with embodiments of the invention are further discussed below.

### Experimental Design Considerations

Metal Pattern Fabrication: Metal patterns may be designed using 2D design tools (e.g., Layout Editor), and an electronic cutter (e.g., Silhouette Cameo 4) may be used to create patterns by cutting a conductive foil. The negative pattern of the metal features may be removed via a tweezer and then transferred to different substrates.

Readout Antenna and IR Fabrication: For readout antenna and IR, copper foil may be used as the conductor with adhesive on the back protected by a glossy paper. After fabrication, the antenna may be transferred to the vinyl, followed by the removal of the glossy paper and pasting on FR-4 substrate (e.g., W/WO copper coated, purchable on Amazon). For the flexible substrate IR, the adhesive side may be covered by another layer of vinyl or polyimide. For transferring the IR onto a conformal surface, the patterned metal may be first transferred onto water-soluble tape, pasted on the desired surface by removal of the glossy paper, and released from the water-soluble tape in water. One layer of vinyl covering may be then used to protect the bare copper traces.

Nutrient Sensor Fabrication: Fabrication and characteristics of the nutrient sensors may be designed using a variety of methods knonw to one of ordinary skill in the art.

Pressure Sensor Fabrication: A patterned 1.5 cm spiral square copper electrode may be pasted on a plastic cover slip and placed on a 3D printed box. After pouring Ecoflex-10 (Smooth On) layers of differing thickness on the top of this electrode, the top electrode (attached to plastic coverslip) may be aligned on the Ecoflex. This setup may be cured at room temperature for about four hours.

Temperature Sensor Fabrication: For the Smartcup pure Polyethylyne Glycol (PEG-1500, Alfa Aesar) solution may be used, while for the wristband PEG-1500 may be diluted in DI water at 1 g/ml concentration. These solutions may be heated at 70° C. and deposited on patterned 1 cm 2.25 turns spiral square copper electrodes. Top electrode layer may be aligned and placed before the temperature could drop. Both electrodes may be attached to a plastic coverslip. The completed sensor may be embedded into an encapsulation layer to prevent the leaking of liquid PEG-1500. This may be created by dipcoating the sensor in multiple layers of Ecoflex-50 (Smooth On).

pH Sensor Fabrication: A p(NIPAM-co-AA) hydrogel may be used as interlayer synthesized by mixing 10% w/v N-Isopropylacrylamide (NIPAM, Sigma), 0.1% w/v methylene bisacrylamide (BIS, Sigma), 0.8% acrylic acid (AA, Sigma), 2.8% v/v N,N,N′,N′-Tetramethyl-ethylenediamine (TEMED, Sigma), and 0.28% w/v ammonium persulfate (APS, Sigma) at OC. The precursor solution may be deposited on 1 cm splitring, and the top splitring layer may be aligned before final gelation. Sensor may be equilibrated in pH 4 buffer solution for at least 24 hours before experimentation.

Salt Sensor Fabrication: Salt sensors may be formed similar to pH sensors, however a PEGDA 700 hydrogel was used for interlayer instead. This may be formed by mixing 10% v/v poly (ethylene glycol) diacrylate (PEGDA 700, Sigma), 0.2% v/v TEMED, and 0.1% w/v APS at 0 C.

Wristband Fabrication: A 3D mold made of PLA may be used to form the base layer of the wristband (Ecoflex-30, Smooth On or PDMS, SYLGARDTM 184, base: curing agent=10:1) with fixed compartments for each sensor. After placing four sensors (pressure, temperature, salt, pH) into their own compartment, another layer of (Ecpflex-30, PDMS-10:1) may be deposited to form the wristband. Temperature and pressure sensors may be sealed within the silicone, however salt and pH sensors possess openings in the bottom layer to enable sweat access.

In-Vitro Characterization: Networks may be probed via either a nanoVNA (NanoRFE) or tabletop VNA (keysight, E5063A). Readout antenna may be aligned against the network as noted in diagram, and spectral response of the network may be probed.

Smartcup Testing: Four sensors (temperature alongside three nutrient sensors: salt-optimized, sugar-optimized, fat-optimized) may be placed on the inside of the cup after fixing and aligning with the 5 cm diameter side of the IR. A 2.5 cm readout antenna may be placed near the 3 cm diameter side of the IR to co-measure sensor response. Sensors were isolated in small compartments to comprehensively validate the lack of cross-coupling in individual sensor readout. First the temperature sensor may be heated to 50° C. and measurements were taken while cooling slowly. Then, 100 g/L glucose (D-Glucose, Sigma) may be added to the sugar-optimized sensor, followed 20 μL oleic acid (Oleic Acid, Fisher Scientific) to the fat-optimized sensor, and finally 25 mg/dL salt (NaCl, Sigma) to the salt-optimized sensor. The total spectra may be monitored throughout this process.

Wristband Testing: Manual pressure may be applied on the pressure sensor, 10 mg/dl NaCl may be added to the salt sensor, deionized water may be added to the pH sensor, while the temperature sensor may be heated to 40° C. and allowed to cool while measurements were taken.

RF Circuit Simulations: For FR circuit simulation Keysight Pathwave Advanced Design System (ADS) may be used for three sensors where synthetic R, L, and C are placed in parallel.

FDTD Simulations: The Finite-difference time-domain (FDTD) may be adopted for EM simulations in the CST microwave studio simulations (RF module). A discreate port and open boundary conditions may be for a hexahedral mesh in the solution. A 1.5 cm×1.5 cm SRR may be coupled to the circular coil for CWOG and CWG. The interlayer of SRR may have a thickness of 1 mm and permittivity of 3. The distance between the sensor and circular antenna is 3 mm. Frequency interval for the Gaussian shaped excitation function ranged from 0-1 GHz. All conducting plates are lossy pure copper with thickness of 50 micron unless otherwise stated. E and H fields may be obtained at normalized maximum color plot.

In the case containing a curving Intermediate Relay (IR) to readout multiple sensors, the arc may have a length of 40 mm and radian of 1 rad. The distance between antenna and IR, IR and sensor may be both 1 mm. Three identical 1.1 cm×1.1 cm, 4.875-turns sensors (strip width and gap are all 0.5 mm) may be aligned along the axis of symmetry. The average magnetic field distribution may be acquired by taking fields on the plane of symmetry.

Although specific network design considerations are discussed above, any of a variety of network design components, configurations, and considerations as appropriate to the requirements of a specific application can be utilized in accordance with embodiments of the invention. While the above description contains many specific embodiments of the invention, these should not be construed as limitations on the scope of the invention, but rather as an example of one embodiment thereof. It is therefore to be understood that the present invention may be practiced otherwise than specifically described, without departing from the scope and spirit of the present invention. Thus, embodiments of the present invention should be considered in all respects as illustrative and not restrictive.

1. A sensor network for spectrometric monitoring of environmental signals, the sensor network comprising:
an RF reader wireless connected to an intermediate relay; and
the intermediate relay coupled to a plurality of passive radio frequency (RF) sensors; and
wherein the plurality of RF sensors are individually programmable for measuring at least one environmental signal.

2. The sensor network of claim 1, wherein the intermediate relay is wirelessly coupled to the plurality of passive RF sensors.

3. The sensor network of claim 1, wherein the sensor network monitors multiple environmental signals within a single spectral readout.

4. The sensor network of claim 1, wherein the plurality of passive RF sensors is an array of passive RF sensors.

5. The sensor network of claim 1, wherein the intermediate relay extends a communicable distance between the RF reader and the plurality of RF sensors.

6. The sensor network of claim 1, wherein the intermediate relay allows for communication over curved surfaces.

7. The sensor network of claim 1, wherein the intermediate relay comprises at least one readout coil.

8. The sensor network of claim 1, wherein the intermediate relay is coupled to the plurality of passive RF sensors via an alignment that remains fixed.

9. The sensor network of claim 1, wherein the intermediate relay is fused onto a textile.

10. The sensor network of claim 1, wherein the plurality of RF sensors are embedded into a wristband.

11. The sensor network of claim 1, wherein the plurality of RF sensors are embedded into a container.

12. The sensor network of claim 11, wherein the container is a cup.

13. The sensor network of claim 1, wherein the at least one environmental signal comprises at least one chemical environmental signal and at least one physical environmental signal.

14. The sensor network of claim 13, wherein the at least one chemical environment signal comprises nutrients.

15. The sensor network of claim 13, wherein the at least one physical environment signal comprises temperature.

16. The sensor network of claim 13, wherein the at least one physical environment signal comprises pressure.

17. The sensor network of claim 13, wherein the at least one chemical environmental signal comprises PH.

18. The sensor network of claim 13, wherein the at least one physical environment signal comprises biometrics.

19. The sensor network of claim 13, wherein the at least one chemical environment signal comprises biometrics.

20. The sensor network of claim 13, wherein a first RF sensor of the plurality of RF sensors is individually programmable for measuring the at least one chemical environmental signal and a second RF sensor of the plurality of RF sensors is individually programmable for measuring the at least one physical environmental signal.
